(19) United States
(12) Patent Application Publication
(10) Pub. No.: US 2025/0032569 A1
(43) Pub. Date: Jan. 30, 2025

(54) COMPOSITION, IN PARTICULAR COSMETIC FORMULATION, COMPRISING A VERBASCOSIDE EXTRACT

(71) Applicant: MEDENA AG, Affoltern am Albis (CH)

(72) Inventors: Wolfgang Mayer, Zürich (CH); Liudmila Korkina, Rome (IT)

(73) Assignee: MEDENA AG, Affoltern am Albis (CH)

(21) Appl. No.: 18/712,962

(22) PCT Filed: Nov. 26, 2021
(86) PCT No.: PCT/EP2021/083201

§ 371 Date: May 23, 2024

(51) Int. Cl. A61K 36/53; A61K 8/60; A61K 8/9789; A61K 31/7032; A61K 36/63; A61K 36/64; A61K 36/80; A61K 36/85; A61Q 17/04

(52) U.S. Cl. CPC A61K 36/53; A61K 8/602; A61K 8/9789; A61K 31/7032; A61K 36/63; A61K 36/64; A61K 36/80; A61K 36/85; A61Q 17/04

(57) ABSTRACT

A composition, in particular a cosmetic formulation, comprising an extract containing verbascoside and/or derivatives thereof and/or structural analogs thereof, wherein the extract is prepared from aerial parts of plants selected from the group consisting of sesame plant (Sesamum indicum), Lippia citriodora, Haeberlea rhodopensis, Cistanche tubulosa, Syringa vulgaris, Aiuga reptens, Buddleija davidii, Verbena officinalis, and Olea europea or wherein the extract is prepared from cell callus cultures of said parts of plants.

### BACKGROUND OF THE INVENTION

The present invention relates to a composition, in particular a cosmetic formulation, comprising an extract containing verbascoside and/or derivatives thereof and/or structural analogs thereof. The invention further relates to a use of such a composition as a topic sunscreen against UV-A and UV-B radiation. Furthermore, the present invention relates to a method for obtaining an extract comprising verbascoside, derivatives thereof and/or structural analogs thereof.

Topical sun protective cosmetics (sunscreens, pre- and post-sun) have been intensively developed and produced to protect human skin against solar radiation-associated damages/pathologies. Unfortunately, known cosmetics for sun protection containing synthetic organic and/or physical sunscreens could have adverse effects on human organisms and contribute to undesirable ecological changes.

The human skin has a particular photo-chemical barrier whose purpose is to detoxify/eliminate solar light-modified substances of low molecular weight.

The outmost external photo-chemical barrier encountered by environmental radiation is the skin surface lipids, a protective hydrophobic film occurring more abundantly in the most photo-exposed cutaneous districts (De Luca, C.; Valacchi, G. Surface lipids as multifunctional mediators of skin responses to environmental stimuli. Mediators Inflamm. 2010, 2010, 321494; doi: 10.1155/2010/321494). The lipid barrier is a mixture of epidermal lipids deriving: (a) from the exfoliating stratum corneum, mainly composed of keratinocyte debris phospholipids and their products of hydrolysis, and (b) from lipids, triglycerides, sterols, and lipophilic vitamins (vitamin E and coenzyme Q10) produced by sebaceous glands. The lipids synthesized in sebocytes are rich in the highly oxidizable triterpenoid squalene, a highly lipophilic molecule present uniquely on human skin, which is regarded not only as a water-insulating and skin-smoothening factor, but most importantly as a sacrificial antioxidant (De Luca, C.; Picardo, M.; Breathnach, A.; Passi, S. Lipoperoxidase activity of Pityrosporum: characterisation of by-products and possible role in Pityriasis versicolor. Exp. Dermatol. 1996, 5, 49-56; Ekanayake Mudiyanselage, S.; Hamburger, M.; Elsner, P.; Thiele, J. J. Ultraviolet A induces generation of squalene monohydroperoxide isomers in human sebum and skin surface lipids in vitro and in vivo. J. Invest. Dermatol. 2003, 120, 915-922). Under UV exposure, following the rapid degradation of the lipophilic antioxidants, squalene remains a main guardian protecting precious epidermal unsaturated phospholipid moieties from the UV-induced free radical-driven oxidative damage. The short-lived, hydrophilic, low-molecular-weight oxidative by-products of squalene oxidation, which are able to rapidly diffuse to viable epidermal and dermal layers, have been proven to be an early signal triggering the adaptive skin immune response to UV radiation (Picardo, M.; Mastrofrancesco, A.; Biro, T. Sebaceous gland—a major player in skin homeostasis. Exp. Dermatol. 2015, 24, 485-486) and a cutaneous metabolic response to solar-simulating UV (Kostyuk, V.; Potapovich, A.; Stancato, A.; De Luca, C.; Lulli, D.; Pastore, S.; Korkina, L. Photo-oxidation products of skin surface squalene mediate metabolic and inflammatory responses to solar UV in human keratinocytes. PLOS One 2012, 7, e44472; doi: 10.1371/journal.pone.0044472). The rate of squalene, vitamin E, and coenzyme Q10 degradation has therefore been proposed as a feasible parameter for measuring the efficacy of sun-protecting formulations (Auffray, B. Protection against singlet oxygen, the main actor of sebum squalene peroxidation during sun exposure, using Commiphora myrrha essential oil. Int. J. Cosmetic Sci. 2007, 29, 1, 23-29). A great majority of synthetic sunscreens do not classify as broadband UV protectors since they can absorb either only UV-B or only UV-A light (FIG. 1).

TABLE 1
Modern synthetic organic sun filters
ORGANIC (SYNTHETIC) CHEMICAL SUNSCREENS
MAINLY ABSORB EITHER UV-B OR UV-A

| | | |
|---|---|---|
| Mexoryl SX | | UV-A |
| Mexoryl XL | UV-B | UV-A |
| Parsol SLX | UV-B | |
| Uvasorb HEB | UV-B | |
| Tinosorb M | UV-B | |
| Neoheliopan AP | | UV-A |
| Tinosorb S | UV-B | UV-A |
| Uvinul A plus | | UV-A |

Usually, to achieve an optimal composition of topical sunscreens labeled as products with high broadband UV-A and UV-B protection, several synthetic molecules having nature-inspired polyphenol moieties (derivatives of benzoic or cinnamic acids) are combined (Wolf, R.; Wolf, D.; Morganti, P.; Ruocco, V. Sunscreens. Clin. Dermatol. 2001, 9, 452-459; Commission recommendation of 22 September 2006 on the efficacy of sunscreen products and the claims made relating thereto. Official J. of the European Union 2006, 1.265, 39-43). To meet requirements of regulatory bodies for claimed SPF-B and SPF-A values, these synthetic substances should be added to sunscreen cosmetics/drugs in high concentrations ranging from 10 to 25% (Commission recommendation of 22 September 2006 on the efficacy of sunscreen products and the claims made relating thereto. Official J. of the European Union 2006, 1.265, 39-43), which sharply increases the risks of adverse cutaneous reactions (inflammation, allergy) to poly-aromatic synthetics (Korkina, L. G.; Pastore, S.; De Luca, C.; Kostyuk, V. A. Metabolism of plant polyphenols in the skin: beneficial versus deleterious effects. Curr. Drug Metab. 2008, 9, 710-729) and might have a negative environmental impact. For example, synthetic sunscreens being endocrine disruptors negatively affect not only humans but also marine and terrestrial species. A large potential portfolio of industrial applications of secondary plant metabolites promises breakthrough sunscreening properties with reduced toxicity and appealing environmental sustainability. The problem of photo-stability of sunscreens has been drawing close attention since quite some time because it might seriously affect desired durable photo-protection and recommendations regarding the frequency of reapplication. Previous studies have shown that synthetic sunscreens lose a significant part of their protection when exposed to UV radiation (Maier, H.; Schauberger, G.; Brunnhofer, K.; Honigsmann, H. Change in ultraviolet absorbance of sunscreens by exposure to solar-simulated radiation. J. Invest. Dermatol. 2001, 117, 256-262; Marrot, L.; Belaidi, J. P.; Lejeune, F.; Maunier, J. R.; Asselineau, D.; Bernerd, F. Photostability of sunscreen products influences the efficiency of protection with regard to UV-induced genotoxic or photoageing-related endpoints. Br. J. Dermatol. 2004, 151, 1234-1244). Unfortunately, many natural UV-A+UV-B screens based on plant extracts and plant-derived oils could also be susceptible to photodegradation mainly by the UV-A range of solar radiation (Bianchi, A.; Marchetti, N.; Scalia, S. Photodegradation of (−)-epigallocatechin-3-gallate in topical cream formulations and its photostabilization. J. Pharm. Biomed. Anal. 2011, 56, 692-697; Kostyuk, V.; Potapovich, A.; Albuhaydar, A. R.; Mayer, W.; De Luca, C.; Korkina, L. Natural substances for prevention of skin photoageing: screening systems in the development of sunscreen and rejuvenation cosmetics. Rejuvenation Res. 2017, Epub ahead of print August 28; doi: 10.1089/rej.2017.1931). Organic (synthetic) and mineral UV filters pose relevant challenges related to their photo-stability in vitro and in vivo under UV radiation (Maier, H.; Schauberger, G.; Brunnhofer, K.; Honigsmann, H. Change in ultraviolet absorbance of sunscreens by exposure to solar-simulated radiation. J. Invest. Dermatol. 2001, 117, 256-262; Marrot, L.; Belaidi, J. P.; Lejeune, F.; Maunier, J. R.; Asselineau, D.; Bernerd, F. Photostability of sunscreen products influences the efficiency of protection with regard to UV-induced genotoxic or photoageing-related endpoints. Br. J. Dermatol. 2004, 151, 1234-1244), their homogeneous distribution onto the skin (Bianchi, A.; Marchetti, N.; Scalia, S. Photodegradation of (−)-epigallocatechin-3-gallate in topical cream formulations and its photostabilization. J. Pharm. Biomed. Anal. 2011, 56, 692-697; Lademann, J.; Rudolph, A.; Jacobi, U.; Weigmann, H. J.; Schaefer, H.; Sterry, W.; Meinke, M. Influence of nonhomogeneous distribution of topically applied UV filters on sun protection factors. J. Biomed. Opt. 2004, 9, 1358-1362; Sohn, M.; Hêche, A.; Herzog, B.; Imanidis, G. Film thickness frequency distribution of different vehicles determines sunscreen efficacy. Biomed. Opt. 2014, 19, 115005; doi: 10.1117/1.JBO.19.11.115005), and their sufficient accumulation in the stratum corneum (Durand, L.; Habran, N.; Henschel, V.; Amighi, K. In vitro evaluation of the cutaneous penetration of sprayable sunscreen emulsions with high concentrations of UV filters. Int. J. Cosmet. Sci. 2009, 31, 279-292) while avoiding their systemic penetration because of the widespread concerns regarding their toxicity (Freitas, J. V.; Praça, F. S.; Bentley, M. V.; Gaspar, L. R. Trans-resveratrol and beta-carotene from sunscreens penetrate viable skin layers and reduce cutaneous penetration of UV-filters. Int. J. Pharm. 2015, 30, 131-137; Stiefel, C.; Schwack, W.; Nguyen, Y.-T. H. Photostability of Cosmetic UV Filters on Mammalian Skin Under UV Exposure. Photochem. Photobiol. 2015, 91, 84-91). In spite of the ongoing intense biological investigations and technological improvements, so far no entirely natural UV filter has been officially approved for the EU market (Annex VI, Directive. EC-1223/2009). For the conventional synthetic organic filters, many of which are potential endocrine disruptors, the inhibition of their deep penetration into the dermis remains a primary clinical goal. This intrinsic property of synthetic organic filters has a major impact on environmental sustainability because of the risk posed by the high concentration of endocrine-disrupting chemical UV-filters in aquatic environments. Concerns are raised though regarding the safety of the long-term application of the novel vehicles and regarding the environmental impact of cosmetic micro- and nano-particles (Baldisserotto, A.; Buso, P.; Radice, M.; Dissette, V.; Lampronti, I.; Gambari, R.; Manfredini, S.; Vertuani, S. Moringa oleifera leaf extracts as multifunctional ingredients for “natural and organic” sunscreens and photoprotective preparations. Molecules 2018, 23 (3). pii E664. doi: 10.3390/molecules23030664).

WO 2018/209449 suggests a use of naturally glycosylated polyphenols, such as verbascoside, as protective agents against the effects of ultraviolet radiation. These known agents comprise glycosylated polyphenols in concentrations of 0.003% to about 6%. The verbascosides contained in such agents may be plant-derived. The plants usually used to obtain verbascoside contain very small amounts of verbascoside. Verbascoside and its analogs typically absorb UV light within the wavelength range of 290 to 400 nm (a broadband UV-B and UV-A range of the solar spectrum). Hence, they could be employed as effective UV-A-UV-B screening molecules. Verbascoside and its analogs can protect skin from deleterious effects of UV-B and UV-A radiation or inhibit skin reactions to this radiation; to name a few, they can prevent/inhibit free radical formation in the skin irradiated by UV-B and UV-A, melanin formation leading to skin tanning, matrix metalloproteinase (MMPs) stimulation, and inflammatory and metabolic reactions in the skin that lead to premature skin photo-ageing.

### SUMMARY OF THE INVENTION

The object of the present invention is to provide a composition with enhanced properties in view of UV-B and UV-A absorption and in protecting the human skin and in view of environmental friendliness.

This object is solved by a composition comprising an extract containing verbascoside and/or derivatives thereof and/or structural analogs thereof, in particular teupolioside and isoverbascoside, wherein the extract is prepared from aerial parts of plants selected from the group consisting of sesame plant (Sesamum indicum), Lippia citriodora, Haeberlea rhodopensis, Cistanche tubulosa, Syringa vulgaris, Aiuga reptens, Buddleija davidii, Verbena officinalis, and Olea europea.

Preferred structural analogs of verbascoside are myconoside, echinacoside, isoverbascoside, 2′-acetyl-verbascoside, cistanoside A, cistanoside C, and tubuloside.

Surprisingly, it has been found that compositions containing extracts from the plants listed as disclosed herein have particularly improved properties regarding the protection of the human skin against solar radiation. These plants have surprisingly shown to contain high concentrations of verbascoside. Because of the high amounts of verbascoside contained in these plants, not further chemical/mineral sunscreens have to be added in order to achieve a highly effective UV-A and UV-B absorption. Particularly advantageous formulations have surprisingly been obtained using the aerial parts of the sesame plant.

Particularly preferably, the content of verbascoside and/or derivatives thereof and/or structural analogs thereof is 6.6 to 25 wt-%. At these verbascoside contents in the composition according to the invention, the same sunscreen effects as with conventional synthetic or physical sunscreens are achieved.

Sun energy in the form of ultraviolet B and A radiation cannot react with the skin surface anymore because verbascoside and its analogs effectively absorb broadband UV-B and UV-A radiation. While effectively absorbing energy of UV-B and UV-A, verbascoside and its analogs cannot be destroyed due to their high photo-stability (sunscreen photo-stability allows less frequent application of sun protective cosmetics to the skin). Sun protective cosmetics containing verbascoside and its analogs as sunscreens effectively rescue endogenous skin antioxidants (vitamin E, coenzyme Q10, and squalene) located in the uppermost skin surface lipids (SSL), thus protecting the native skin photo-barrier. These sun protective cosmetics are safe for human beings (absence of toxicity, photo-toxicity, and photo-allergenicity) and the marine and terrestrial environment.

Phenylethanoid/phenylpropanoid glycosides are naturally occurring water-soluble compounds. Structurally, they are characterized by cinnamic acid (C6-C3) and hydroxyphenylethyl (C6-C2) moieties that are attached to a β-glucopyranose (apiose, galactose, rhamnose, xylose, etc.) via a glycosidic bond. In recent years, interest has been growing regarding plant-derived aromatic compounds, and phenylethanoid glycosides in particular, because of the tremendously growing volume of literature describing their evident role in the prevention and treatment of various human diseases and disorders.

Verbascoside (syn. acteoside, kusaginin, orobanchin) is a caffeoyl phenylethanoid glycoside, in which the phenylpropanoid caffeic acid and the phenylethanoid hydroxytyrosol are connected by the ester bond. Another ether bond connects them to the rhamnose and the glucose (disaccharide). The molecule formed has the chemical name β-(3′,4′-dihydroxyphenyl)ethyl-O-α-L-rhamnopyranosyl (1->3)-β-D-(4-O-caffeoyl)-glucopyranoside and the traditional name verbascoside (recommended use instead of syn. acteoside, kusaginin, orobanchin) (Alipieva et al., 2014). The chemical structure of verbascoside is shown in FIG. 1.

Preferably, the composition of the invention comprises at least one compound selected from the group consisting of amino acids, fatty acids, polysaccharides, sterols, vitamins, minerals, and phytochemical compounds.

Normally, the composition according to the invention has the form of a cream, a milky emulsion or a transparent lotion or a serum or a spray. Preferably, the composition according to the invention is characterized by an in-vitro SPF equal to or higher than 20. Preferably, the composition according to the invention shows an SPF-B from 20 to 50+. The extract of the composition according to the invention is qualitatively analyzed by UV-vis spectrometry, high-performance liquid chromatography (HPLC) or a combination thereof using commercial standards of verbascoside, isoverbascoside, and teupolioside. Advantageously, the verbascoside is embedded by liposomes, lipogels, hydrogels, nanoparticles or any other intracutaneous carrier. Skin permeation and the transdermal delivery of verbascoside can be increased substantially by its inclusion into liposomes or lipogels. Advantageously, the composition according to the invention is characterized by water resistance and adhesion to the skin surface.

The present invention further relates to a composition according to the invention as a topic sunscreen against UV-A and UV-B radiation.

The present invention also relates to a method for obtaining an extract comprising verbascoside, derivatives thereof and/or structural analogs thereof, such as teupolioside, isoverbascoside, 2′-acyl-verbascoside, echinacoside, myconoside, tubuloside C, cistanoside A, and cistanoside C, the process comprising the steps of:
a) selecting at least one aerial part or a callus cell culture of a plant selected from the group consisting of sesame plant (Sesamum indicum), Lippia citriodora, Haeberlea rhodopensis, Cistanche tubulosa, Syringa vulgaris, Aluga reptens, Buddleija davidii, Verbena officinalis, and Olea europea;
b) extracting verbascoside, derivatives thereof and/or structural analogs thereof present in the plant part via a technique selected from the group consisting of washing, decoction, maceration, homogenization, percolation, and any combination thereof;
c) separating the main liquid phase which comprises the extracted compounds from the solids greater than approximately 2 mm in size, by natural sedimentation, filtration, centrifugation, or a combination thereof;
d) clarifying the liquid phase obtained in step c); and
e) concentrating the clarified liquid phase.

Preferably, the method according to the invention comprises an additional step of pretreating the aerial plant part before the extraction step b), wherein the pretreatment comprises drying the aerial plant part to a moisture content of less than 80% to 60% and/or chopping the plant waste part to a size less than 5 cm.

Furthermore, the extraction step of the method according to the invention is carried out in water or a solvent/water mixture, wherein the solvent is chosen from the group consisting of methanol, ethanol, acetone, and ethyl acetate.

The extraction step of the method according to the invention is preferably carried out at a temperature between 25° C. and 90° C., wherein the extraction step is preferably carried out for 20 to 60 minutes, wherein the extraction step is preferably carried out under continuous magnetic stirring or vortexing.

Preferably, the solids separated out in step c) of the method according to the invention are subjected to a second extraction under the same conditions as the extraction performed in step b), wherein the secondary liquid phase is separated from the solids greater than 2 mm in size, and wherein the secondary liquid phase is mixed with the main liquid phase previously obtained.

Preferably, the method according to the invention comprises an additional step f), in which the concentrated liquid from step e) is transformed into powder via a spray-drying technique.

Preferably, the aerial plant parts are plant waste derived from the industrial manufacturing of plant-based food products, such as sesame oil, olive oil or sesame bran.

### BRIEF DESCRIPTION OF THE DRAWINGS

FIG. 1: Chemical structure of verbascoside

FIG. 2: Representative absorption spectra of aqueous Haberlea rhodopensis solutions containing myconoside

FIG. 3: Representative absorption spectra of aqueous Haberlea rhodopensis with 0.001% of myconoside without or after 2 min, 10 min, 20 min, and 40 min of UV administration

FIG. 4: Representative absorption spectra of Cistanche tubulosa extract

FIG. 5: Representative absorption spectra of 0.002% water solutions of Cistanche tubulosa extracts without and after 40 min UV-irradiation

FIG. 6: Representative absorption spectra of aqueous Lippia citriodora solutions containing 40% of verbascoside.

FIG. 7: Representative absorption spectra of 0.002% water solutions of Lippia citriodora extract without and after 5, 10 20, and 40 min UV-irradiation

FIG. 8: Absorption spectrum of 0.01% water/alcoholic extracts from various plant materials (vigorous vortexing for 20 min)

FIG. 9: Absorption spectrum of 0.02% extracts of Sesamum indicum leaves by water or ethanol and of 0.01% extract by water/ethanol mixture subjected to different extraction time

FIG. 10: Absorption spectrum of 0.02% extracts of Olea europea leaves by water or ethanol and of 0.01% extract by water/ethanol mixture subjected to different extraction time

FIG. 11: Absorption spectrum of 0.02% extracts of Olea europea fruit mash by water or ethanol and of 0.01% extract by water/ethanol mixture subjected to different extraction times

FIG. 12: Dependence of SPF values on the initial amount of ground sesame leaves

FIG. 13: Absorption spectrum of 0.001% solutions of liquid sunscreens

FIG. 14: Dose-dependent curve of SPF values

FIG. 15: Dose-dependent curve of SPF-B values

FIG. 16: Dose-dependent curve of SPF-B values

FIG. 17: Absorption spectrum of 0.001% solutions of powder sunscreens

FIG. 18: Dose-dependent curve of SPF-B values

FIG. 19: Dose-dependent curve of SPF-B values

FIG. 20: Dose-dependent curve of SPF-B values

FIG. 21: Absorption spectrum of 0.0005% heptane solutions of bis-ethylhexyloxyphenol methoxyphenyl triazine

FIG. 22: Dose-dependent curve of SPF-B values

FIG. 23: Representative absorption spectra of water solutions of chemicals without and after 40 min UV-irradiation.

FIG. 24: UV-visible absorption spectra of water/ethanol (1:1 w/w) solution of samples (0.5 mg/ml)

FIG. 25: Representative absorption spectra of (0.5 mg/ml) water/alcohol solutions of Lotion SPF 30 (A), Lotion SPF 50 (B) and Cream SPF 50 (C) without (0 min) and after 40 min UV-irradiation

FIG. 26: Representative absorption spectra of lotions containing mixtures of verbascoside- and myconoside-containing extracts at concentrations 5%/5% (first/topmost line), 4%/4% (second line), 3.5%/3.5% (third line, and 2%/2% (fourth/lowermost line). Lotions were diluted 2,500 times by alcohol: water (4:1 w/w) solutions for spectrophotometry

FIG. 27: HPLC analysis of Cistanche tubulosa extract

### DETAILED DESCRIPTION

### Examples

Five examples of compositions according to the invention are presented in the tables below.

### Example 1

| INCI/CTFA name | % |
|---|---|
| AQUA/[WATER] | 64.22800000 |
| GLYCINE SOJA OIL/[GLYCINE SOJA | 13.50000000 |
| (SOYBEAN) OIL] | |
| VERBASCOSIDE | 12.00000000 |
| GLYCERYL ROSINATE | 3.80000000 |
| GLYCERYL STEARATE CITRATE | 2.00000000 |
| CETEARYL ALCOHOL | 1.00000000 |
| CETYL ALCOHOL | 0.90000000 |
| XANTHAN GUM | 0.70000000 |
| SODIUM STEAROYL GLUTAMATE | 0.50000000 |
| LONICERA CAPRIFOLIUM FLOWER | 0.45000000 |
| EXTRACT/[LONICERA CAPRIFOLIUM | |
| (HONEYSUCKLE) FLOWER EXTRACT] | |
| TOCOPHEROL | 0.40000000 |
| LONICERA JAPONICA FLOWER | 0.20000000 |
| EXTRACT/[LONICERA JAPONICA | |
| (HONEYSUCKLE) FLOWER EXTRACT] | |
| DISODIUM PHOSPHATE | 0.10000000 |
| TETRASODIUM GLUTAMATE DIACETATE | 0.09800000 |
| CELLULOSE | 0.08000000 |
| SODIUM SULFATE | 0.04000000 |
| SODIUM HYDROXIDE | 0.00400000 |
| Total | 100.0 |

### Example 2

| INCI/CTFA name | % |
|---|---|
| AQUA/[WATER] | 51.67300000 |
| GLYCINE SOJA OIL/[GLYCINE SOJA | 12.00000000 |
| (SOYBEAN) OIL] | |
| VERBASCOSIDE | 12.00000000 |
| CETEARYL ALCOHOL | 6.20000000 |
| GLYCERYL STEARATE | 4.00000000 |
| COCONUT ALKANES | 3.65000000 |
| BUTYROSPERMUM PARKII | 3.00000000 |
| BUTTER/[BUTYROSPERMUM PARKII | |
| (SHEA BUTTER)] | |
| GLYCERIN | 3.00000000 |
| COCO-CAPRYLATE/CAPRATE | 1.25000000 |
| CETEARYL GLUCOSIDE | 0.80000000 |
| SODIUM STEAROYL GLUTAMATE | 0.50000000 |
| LONICERA CAPRIFOLIUM FLOWER | 0.45000000 |
| EXTRACT/[LONICERA CAPRIFOLIUM | |
| (HONEYSUCKLE) FLOWER EXTRACT | |
| TOCOPHEROL | 0.40000000 |
| PANTHENOL | 0.37500000 |
| XANTHAN GUM | 0.30000000 |
| LONICERA JAPONICA FLOWER | 0.20000000 |
| EXTRACT/[LONICERA JAPONICA | |
| (HONEYSUCKLE) FLOWER EXTRACT] | |
| DILINOLEIC ACID/PROPANEDIOL | 0.10000000 |
| COPOLYMER | |
| TETRASODIUM GLUTAMATE | 0.09800000 |
| DIACETATE | |
| SODIUM HYDROXIDE | 0.00400000 |
| Total | 100.0 |

### Example 3

| INCI/CTFA name | % |
|---|---|
| AQUA/[WATER] | 56.97300000 |
| GLYCINE SOJA OIL/[GLYCINE SOJA | 12.00000000 |
| (SOYBEAN) OIL] | |
| VERBASCOSIDE | 6.70000000 |
| GLYCERYL STEARATE | 4.00000000 |
| COCONUT ALKANES | 3.65000000 |
| CETEARYL ALCOHOL | 3.20000000 |
| BUTYROSPERMUM PARKII | 3.00000000 |
| BUTTER/[BUTYROSPERMUM PARKII | |
| (SHEA BUTTER)] | |
| GLYCERIN | 3.00000000 |
| THEOBROMA CACAO SEED | 3.00000000 |
| BUTTER/[THEOBROMA CACAO | |
| (COCOA) SEED BUTTER] | |
| COCO-CAPRYLATE/CAPRATE | 1.25000000 |
| CETEARYL GLUCOSIDE | 0.80000000 |
| SODIUM STEAROYL GLUTAMATE | 0.50000000 |
| LONICERA CAPRIFOLIUM FLOWER | 0.45000000 |
| EXTRACT/[LONICERA CAPRIFOLIUM | |
| (HONEYSUCKLE) FLOWER EXTRACT] | |
| TOCOPHEROL | 0.40000000 |
| PANTHENOL | 0.37500000 |
| XANTHAN GUM | 0.30000000 |
| LONICERA JAPONICA FLOWER | 0.20000000 |
| EXTRACT/[LONICERA JAPONICA | |
| (HONEYSUCKLE) | |
| DILINOLEIC ACID/PROPANEDIOL | 0.10000000 |
| COPOLYMER | |
| TETRASODIUM GLUTAMATE | 0.09800000 |
| DIACETATE | |
| SODIUM HYDROXIDE | 0.00400000 |
| Total | 100.0 |

### Example 4

| INCI/CTFA name | % |
|---|---|
| AQUA/[WATER] | 68.59300000 |
| GLYCINE SOJA OIL/[GLYCINE SOJA | 11.00000000 |
| (SOYBEAN) OIL] | |
| VERBASCOSIDE | 6.70000000 |
| GLYCERYL ROSINATE | 3.80000000 |
| COCO-CAPRYLATE | 3.00000000 |
| GLYCERYL STEARATE CITRATE | 2.00000000 |
| CETEARYL ALCOHOL | 1.50000000 |
| CETYL ALCOHOL | 1.00000000 |
| XANTHAN GUM | 0.70000000 |
| SODIUM STEAROYL GLUTAMATE | 0.50000000 |
| LONICERA CAPRIFOLIUM FLOWER | 0.45000000 |
| EXTRACT/[LONICERA CAPRIFOLIUM | |
| (HONEYSUCKLE) FLOWER EXTRACT] | |
| LONICERA JAPONICA FLOWER | 0.20000000 |
| EXTRACT/[LONICERA JAPONICA | |
| (HONEYSUCKLE) FLOWER EXTRACT] | |
| LECITHIN | 0.10500000 |
| DISODIUM PHOSPHATE | 0.10000000 |
| TETRASODIUM GLUTAMATE DIACETATE | 0.09800000 |
| CITRIC ACID | 0.08500000 |
| CELLULOSE | 0.08000000 |
| SODIUM SULFATE | 0.04000000 |
| ASCORBYL PALMITATE | 0.03750000 |
| TOCOPHEROL | 0.00750000 |
| SODIUM HYDROXIDE | 0.00400000 |
| Total | 100.0 |

### Example 5

| INCI/CTFA name | % |
|---|---|
| AQUA/[WATER] | 64.72800000 |
| GLYCINE SOJA OIL/[GLYCINE SOJA | 13.00000000 |
| (SOYBEAN) OIL] | |
| GLYCERYL STEARATE | 5.00000000 |
| HABERLEA RHODOPENSIS CALLUS EXTRACT | 4.00000000 |
| SYRINGA VULGARIS LEAF CELL | 4.00000000 |
| CULTURE EXTRACT | |
| GLYCERYL ROSINATE | 3.80000000 |
| GLYCERYL STEARATE CITRATE | 2.00000000 |
| CETYL ALCOHOL | 0.90000000 |
| XANTHAN GUM | 0.70000000 |
| SODIUM STEAROYL GLUTAMATE | 0.50000000 |
| LONICERA CAPRIFOLIUM FLOWER | 0.45000000 |
| EXTRACT/[LONICERA CAPRIFOLIUM | |
| (HONEYSUCKLE) FLOWER EXTRACT] | |
| TOCOPHEROL | 0.40000000 |
| LONICERA JAPONICA FLOWER | 0.20000000 |
| EXTRACT/[LONICERA JAPONICA | |
| (HONEYSUCKLE) FLOWER EXTRACT] | |
| DISODIUM PHOSPHATE | 0.10000000 |
| TETRASODIUM GLUTAMATE DIACETATE | 0.09800000 |
| CELLULOSE | 0.08000000 |
| SODIUM SULFATE | 0.04000000 |
| SODIUM HYDROXIDE | 0.00400000 |
| Total | 100.0 |

Examples of extracts according to the invention and known chemical sunscreens:

A. Alcohol/Water Extracts of Haberlea rhodopensis Cell Cultures Containing 36% of Myconoside as Broadband UVB+UVA Protectors with High Photo-Stability and Low Cyto-Photo-Toxicity

Haberlea rhodopensis Friv. (Order: Lamilae; Family: Gesneriaceae) is a rare endemic and preglacial relict growing on the Balkan Peninsula, mainly in the Rhodope mountains in Bulgaria and Greece. H. rhodopensis is a flowering plant, which is highly tolerant to desiccation by freezing and draught. H. rhodopensis is called a resurrection plant because it is able to revive upon rehydration even after prolonged periods (hundreds of years) of complete dehydration. The leaves of H. rhodopensis were used in folk medicine as an anti-inflammatory remedy and for wound healing acceleration. The tea based on H. rhodopensis leaves and flowers is commonly used for energizing, anti-ageing, and rehabilitative purposes. Aqueous/alcoholic extracts of aerial parts of H. rhodopensis plants and of plant cell cultures improve skin elasticity.

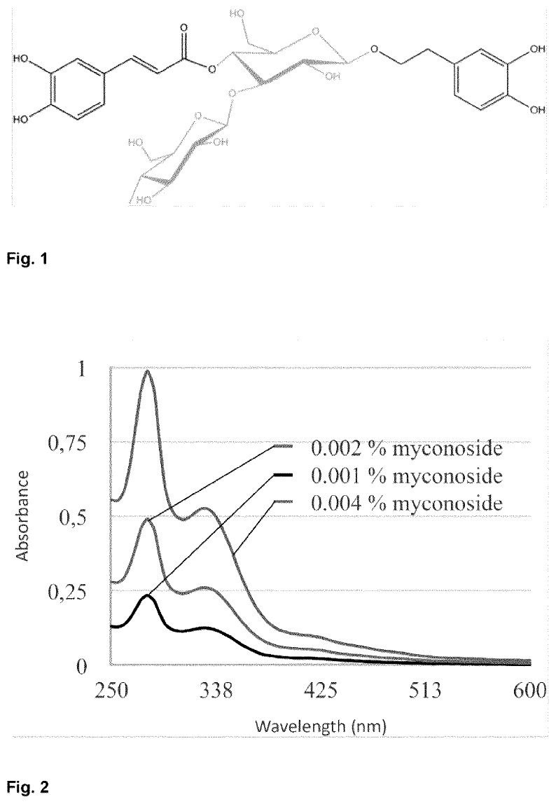

The main biologically active molecules identified in H. rhodopensis are the phenylethanoid glycosides myconoside, paucifloside, and verbascoside. Myconoside [β-(3,4-dihydroxyphenyl)-ethyl-3,6-di-O-β-D-apifuranosyl-4-O-α,β-dihydrocaffeoyl-O-β-D-glucopyranoside] consists of 3,4-dihydroxyphenyl moiety attached to the main sugar glucose, dihydrocaffeoyl structure linked to position C-4 of glucose and two β-apiosyl moieties linked to position C-3 and C-6 of glucose. The myconoside content in aerial parts of H. rhodopensis could reach 88.8% of all polyphenolic secondary metabolites. In absolute values, the content of myconoside in the wild grown plant is approximately 6.5 mg/g dry weight, while its amount in the in vitro cultivated plant cells could reach 84-87 mg/g of dry weight (Amirova, K. M. et al. Biotechnologically-produced myconoside and calceolarioside E induce Nrf2 expression in neutrophils. Int J Mol Sci 2021, 22 (4), 1759). The chemical structure of myconoside is similar to that of verbascoside (See FIG. 1 and Table 2 of Cistanche tubulosa sub-chapter). In the positions R2 and R5 of myconoside molecule, there are 2 D-apifuranosyl sugar moieties.

Goal of the study was to determine whether H. rhodopensis cell culture alcohol/water extract containing 36% of verbascoside possesses natural SPF-B and SPF-A comparable with SPFs of synthetic sunscreens widely used in sun protective cosmetics using the in vitro spectrophotometric methods to predict results of in vivo human studies corresponding the requirements of EU Commission for sun protective cosmetics (COLIPA) (Matts P J, et al. COLIPA in vitro UVA method: a standard and reproducible measure of sunscreen UVA protection. Int J Cosmet Sci 2010, 32 (1); 35-46; COLIPA (European Cosmetics Association) method for the in vitro determination of UVA protection provided by sunscreen products (2009)).

### Sample Preparation for Spectrophotometry:

In vitro cell cultures of H. rhodopensis derived from the plant leaves were grown on a plant growth medium in accord with the method described (Amirova, K. M. et al. Biotechnologically-produced myconoside and calceolarioside E induce Nrf2 expression in neutrophils. Int J Mol Sci 2021, 22 (4), 1759), and were subcultured every 2 months. The 2-months-old in vitro cell cultures of H. rhodopensis were freeze-dried and extracted with 50% aqueous/ethanol under sonication for 20 min at room temperature. The extracts were filtrated, concentrated under a vacuum at 40° C., lyophilized until dryness, and stored at −20° C. before use. The content of myconoside was determined by HPLC using commercially available myconoside as a standard.

Sample of H. rhodopensis extract (36% myconoside) obtained by the proposed method was diluted by distilled water to prepare 1%, 2.5%, 5.0%, 7.5%, 10%, and 20% solutions.

### Spectrophotometry of Samples:

Each sample (1 ml) was placed in a 1 cm quartz cell. The absorbance was measured between 250-600 nm at an interval of every 1 nm using a Varian UV/Vis spectrophotometer (Cary 50 Scan). The following parameters were measured and calculated for each sample assuming standard application to the skin—2 mg/cm²:
the SPF values.
the critical wavelength λ_{C}
the UVA/UVB ratio
   1. Determination of SPF Values of the Aqueous Solutions of Haberlea rhodopensis

A series of water solutions of extracts from Haberlea rhodopensis with concentrations of myconoside from 0.001 to 0.004% were prepared in duplicate and UV-visible absorption spectra of were recorded over the range of 250-600 nm (FIG. 2). From these spectra, the transmission of the sunscreen (T) was found from monochromatic absorbance values at wavelength λ: T=10^{-A(λ)}.

There are two distinguished peaks in aqueous Haberlea rhodopensis solutions with major phenyl-propanoid glycoside-myconoside at 282 nm and 328-330 nm, amplitudes of which depend on myconoside concentration in a linear manner.

The SPF values of aqueous solutions of Haberlea rhodopensis with definite concentrations of myconoside were calculated using Eq 1 for two application conditions: the solution applied at 2 mg/cm² (standard application) and 10 mg/cm². Calculated data (Mean±SD) are shown in Table 2. In the same Table 2, critical wavelength and the UVA/UVB ratios are shown.

TABLE 2
SPF values, critical wavelength (λ_{C}) and UVA/UVB ratio for aqueous solutions
of Haberlea rhodopensis with definite concentrations of myconoside

| | Amount of myconoside | | | Critical | |
|---|---|---|---|---|---|
| | in aqueous solutions | SPF values | | wavelength | |
| No. | of Haberlea rhod. | 2 mg/cm² | 10 mg/cm² | (λ_{C}) | UVA/UVB |
| 1 | 0.05% | 1.03 | 1.14 | 371 nm | 0.58 |
| 2 | 0.1% | 1.06 | 1.32 | 372 nm | 0.58 |
| 3 | 0.2% | 1.11 | 1.71 | 372 nm | 0.58 |
| 4 | 0.25% | 1.12 | 1.75 | 371 nm | 0.58 |
| 5 | 0.5% | 1.25 | 3.0 | 371 nm | 0.57 |
| 6 | 1.0% | 1.6 | 10.8 | 372 nm | 0.58 |
| 7 | 2.5% | 3.0 | 119 | 371 nm | 0.57 |
| 8 | 5.0% | 11 | 974 | 371 nm | 0.57 |
| 9 | 7.5% | 27 | ND | 371 nm | 0.57 |
| 10 | 10.0% | 60 | ND | 371 nm | 0.57 |

Critical Wavelength (λ_{C}) and the UVA/UVB ratios were also calculated from the absorption spectra and were collected in Table 2. According to Boots the Chemist Ltd, a low UVA protection corresponds to a UVA/UVB ratio <0.2 (0 UVA Star Rating), a moderate protection is at a ratio between 0.21 and 0.40 (1 UVA Star Rating), a good protection is at a ratio between 0.41 and 0.6 (2 UVA Star Rating), a superior protection is at a ratio between 0.61 and 0.8 (3 UVA Star Rating) and a maximum protection is at a ratio >0.8 (4 UVA Star Rating). According to these International indications, aqueous solutions of Haberlea rhodopensis with different concentrations of myconoside can be attributed to the group of good protection (2 UVA Star Rating).

2. Photo-Stability of Aqueous Solutions of Haberlea rhodopensis with Myconoside Exposed to Solar Imitating UV Irradiation for 2-40 Minutes

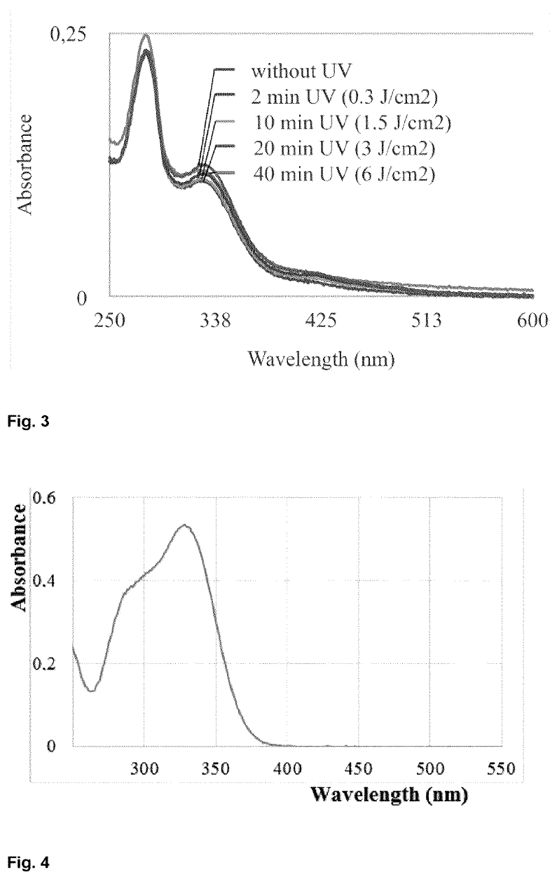

In these experiments, UV-visible absorption spectra of aqueous solution of Haberlea rhodopensis with 0.001% of myconoside were recorded over the range of 250-450 nm without or after 2 to 40 min UV irradiation (FIG. 3). The total dose was 4.8 (UVB 1.6+UVA 3.2) J/cm². It was found that solutions are not destroyed by (UVA+UVB) light. UV irradiation did not affect SPF values of the solutions amid the absorption spectra even after 40 min of irradiation exhibit no changes (FIG. 3).

### Conclusions

1. Protection from UVB irradiation reached SPF 11 at 5% of myconoside, SPF 27 at 7.5% of myconoside, and SPF 60 at 10.0% of myconoside calculated for 2 mg/cm² of application. When application density was increased up to 10 mg/cm², SPF values were dramatically increased.
2. Protection from UVA irradiation assessed by the UVA/UVB ratio and critical wavelength corresponds to good protection UVA 2 Star Rating (in accordance with the International Standards) for the whole range of myconoside concentrations studied (0.05%-10%) and assessed by the critical wave length (>370 nm)
3. Photo-stability of aqueous solutions of Haberlea rhodopensis extracts with 0.001% of myconoside, as a unique natural photo-protective factor, was very high because myconoside was not destroyed by intensive solar imitating UVB+UVA irradiation for at least 40 minutes. So the application of myconoside-containing sun protective products should not be repeated very often.
   B. Aqueous and Alcohol/Water Extracts of Cistanche Tubulosa as Broadband UVB+UVA Protectors with High Photo-Stability and Low Photo-Cyto-Toxicity

Cistanche tubulosa (Schenk) R. Wight is a plant that parasitizes the roots of Tamarix. It grows by absorbing the nutrition out of plants it grows on. It belongs to Cistanche, Orobanchaceae. Cistanche tubulosa grows in the Takla Makan Desert in Hsinchiang Uighur Autonomous Region, China. It has a very strong capacity to flower and fruit under severe desert conditions.

In China, Cistanche tubulosa is known as a rare Panax ginseng found in deserts and used as a pharmaceutical to cure Alzheimer's disease. In Japan however, Cistanche tubulosa has been determined as a food.

According to the Chinese Comprehensive Pharmaceutical Dictionary, Cistanche tubulosa improves renal function, increases sexual power, and smoothes the intestines. The Dictionary also teaches that it treats impotence, infertility, menstrual disorder, and pain of the back and knees. Recent research has discovered that ethanol-aqueous extract of Cistanche tubulosa given orally to experimental animals exhibits age-preventing effects towards skin and brain, prevents age-related fatigue, accelerates fat metabolism, and boost immune system.

Major active components (secondary plant metabolites) in Cistanche tubulosa are phenylpropanoid/phenylethanoid glycosides, especially verbascoside and its close analogues, such as echinacoside, isoverbascoside, 2′-acetyl-verbascoside, cistanoside A, cistanoside C, and tubuloside A. In total, they comprise more than 93% of all polyphenolic secondary metabolites in Cistanche tubulosa. Tiny differences in their chemical structure are present in Table 3.

TABLE 3
Chemical structures of phenylpropanoid glycosides
found in Cistanche tubulosa

| Compound | R1 | R2 | R3 | R4 | R5 |
|---|---|---|---|---|---|
| Verbascoside | H | Rha | Cf | H | OH |
| 2′-acetylverbascoside | Ac | Rha | Cf | H | OH |
| Isoverbascoside | H | Rha | H | Cf | OH |
| Echinacoside | H | Rha | Cf | Glc | OH |
| Cistanoside A | H | Rha | Cf | Glc | OMe |
| Cistanoside C | H | Rha | Cf | H | OMe |
| Tubuloside A | Ac | Rha | Cf | Glc | OH |

R1-R5 are substitutes in the verbascoside chemical structure. Abbreviations: Ac—acetyl; Glc—β D-glucopyranose; Cf—trans-caffeoyl; and Rha—α L-rhamnopyranose.

Isoverbascoside is an optical isomer of verbascoside.

The HPLC analysis of Cistanche tubulosa extract obtained by the proposed method showed that verbascoside (Vb) content was equal 50.3%, echinacoside (Ech) content was equal 7.3%, isoverbascoside (Ivb) content was 18.9%, and 2′-acetylverbascoside (Avb) content was 4.5%

Goal of the study was to determine whether Cistanche tubulosa alcohol/water extract containing 50% of verbascoside possesses natural SPF-B and SPF-A comparable with SPFs of synthetic sunscreens widely used in sun protective cosmetics using the in vitro spectrophotometric methods to predict results of in vivo human studies corresponding the requirements of EU Commission for sun protective cosmetics (COLIPA).

### Sample Preparation for Spectrophotometry:

Sample of Cistanche tubulosa extract 100 μl (0.1 ml) was dissolved in 20 ml distilled water to obtain 5 μl/ml extract/water or 5 mg/ml. Sample (1 ml) of Cistanche tubulosa mixture after 1st dilution (5 μl/ml extract/water) was mixed with 19 ml distilled water to obtain a solution containing 0.25 mg/ml Cistanche tubulosa extract.

### Spectrophotometry of Samples:

Each sample (1 ml) was placed in a 1 cm quartz cell. The absorbance was measured between 250-600 nm at an interval of every 1 nm using a Varian UV/Vis spectrophotometer (Cary 50 Scan). The following parameters were measured and calculated for each sample assuming standard application to the skin—2 mg/cm²:
the SPF values.
the critical wavelength λ_{C}
the UVA/UVB ratio

The SPF values in vitro were calculated according to the method described by Sayre et al., (Sayre, R M; Agin, P P; LeVee, G J; Marlowe, E. A comparison of in vivo and in vitro testing of sunscreening formulas, Photochem. Photobiol. 1979, 29, 559-566). The calculation was done using Equation 1:

$$SPF = {\sum }_{290}^{400} Ser ( λ ) \times Ss ( λ ) / {\sum }_{290}^{400} Ser ( λ ) \times Ss ( λ ) \times T ( λ ) \qquad ( 1 )$$

where Ss(λ) is the spectral irradiance, Ser(λ) is the CIE erythema action spectrum and T(λ) is the transmission of the sunscreen. The values of Ss(λ) and Ser(λ) were taken from the literature (B. L. Diffey, J. Robson. A new substrate to measure sunscreen protection factors throughout the ultraviolet spectrum, J. Soc. Cosmet. Chem. 1989, 40, 127-133).

The Critical Wavelength λ_{C} is the wavelength, where the area under the absorbance spectrum for the irradiated product from 290 nm to λ_{C} is equal to 90% of the integral value of the absorbance spectrum from 290 to 400 nm and was calculated using Eq. 2:

$${\int }_{290}^{λ c} A ( λ ) d λ = 0.9 {\int }_{290}^{400} A ( λ ) d λ \qquad ( 2 )$$

where A (λ)=monochromatic absorbance values at wavelength λ. The critical wavelength was determined for all samples in order to assess a degree of UVA protection. In cases where the critical wavelength is lower than 370 nm, the protection from UVA is less evident.

In order to further characterize solutions as skin photo-protectors from UVA, the UVA/UVB ratio was calculated using Eq. 3:

$$UVA / UVB = {\int }_{320}^{400} A ( λ ) d λ / {\int }_{290}^{320} A ( λ ) d λ \qquad ( 3 )$$

### Determination of UV-Stability:

Stability of solutions under exposure to solar imitating (UVB+UVA) irradiation was recorded spectrophotometrically as follows: samples containing 3 ml of aqueous Cistanche tubulosa solutions with final concentration of 0.002% were exposed to 2 mW/cm² UV light (G6T5E UV-B lamp, emitted ultraviolet rays between 280 nm and 360 nm (at peak 306 nm) UVB 0.7 mW/cm²+UVA 1.3 mW/cm²) in 3.5-cm Petri dishes. The distance from the sample surface was 5.5 cm, the layer thickness was approx. 3 mm. The duration of exposure was 40 min. Irradiance was measured using UVX Digital Radiometer equipped with UVX-31 300 nm and UVX-36 365 nm sensors (Canadawide Scientific). Then, absorbance of the solutions was measured between 250-450 nm using a 1 cm quartz cell. The control samples were measured in the same manner except UV irradiation.

### Results

SPF values were calculated using the spectra and Eq. 1 for following solutions containing Cistanche tubulosa powder at 2%, 5%, 15%, 30% and 40%. The application of these solutions was assumed at 2 mg per cm² of the skin (standard application).

Critical Wavelength (λ_{C}) and UVA/UVB ratio of solutions with Cistanche tubulosa powder were also calculated from the absorption spectra and are given in FIG. 4. According to Boots the Chemist Ltd, a low UVA protection corresponds to a UVA/UVB ratio <0.2 (0 UVA Star Rating), a moderate protection to a ratio between 0.21 and 0.40 (1 UVA Star Rating), a good protection to a ratio between 0.41 and 0.6 (2 UVA Star Rating), a superior protection to a ratio between 0.61 and 0.8 (3 UVA Star Rating) and a maximum one to a ratio >0.8 (4 UVA Star Rating). Therefore, solutions containing different concentrations of Cistanche tubulosa powder can be attributed to the group of good protection (2 UVA Star Rating).

TABLE 4
SPF values, critical wavelength (λ_{C}) and the
UVA/UVB ratio for the solutions containing different
concentrations of Cistanche tubulosa powder

| | Content of | | Critical | |
|---|---|---|---|---|
| | Cistanche tubulosa | SPF | wavelength | |
| No. | extract in solutions (%) | values | (λ_{C}) | UVA/UVB |
| 1 | Solution 2% | 2.6 | 352 mm | 0.51 |
| 2 | Solution 5% | 5.9 | 352 mm | 0.51 |
| 3 | Solution 15% | 35 | 352 mm | 0.51 |
| 4 | Solution 30% | 60 | 352 mm | 0.51 |
| 5 | Solution 40% | 81 | 352 mm | 0.51 |

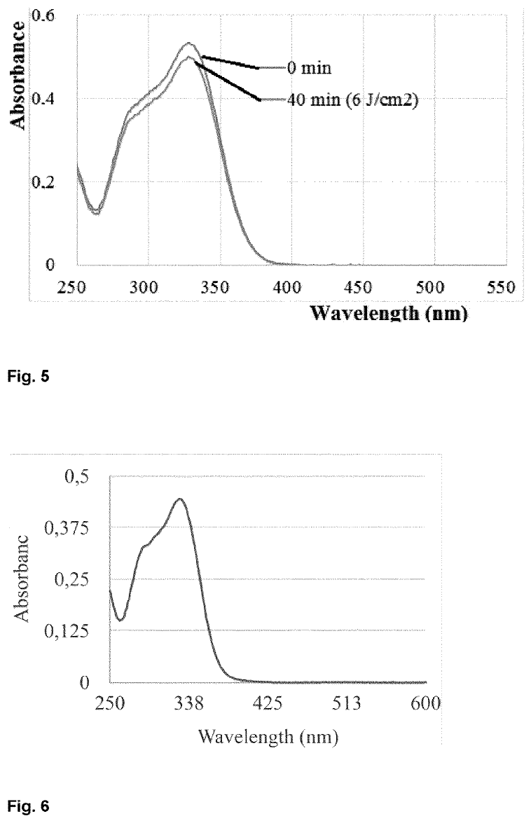

Photo-stability of the Cistanche tubulosa powder in final solutions expressed in SPF values is shown in the following FIG. 5 and Table 5.

TABLE 5
Photo-stability of Cistanche tubulosa
powder suspension expressed in SPF values

| Amount of Cistanche | Initial SPF values | SPF values after |
|---|---|---|
| tubulosa extract in | without exposure to | 40 min UVA + UVB |
| final solutions (%) | UVA + UVB | exposure |
| 2 | 2.6 | 2.5 |
| 5 | 5.9 | 5.8 |
| 15 | 35 | 34 |
| 30 | 60 | 60 |
| 40 | 81 | 80 |

C. Tests for UVB Protection (Sun Protection Factor (SPF)), UVA Protection (UVA/UVB), and Photo-Stability of Aqueous Solutions of Lippia citriodora Extracts

Extracts of Lippia citriodora (a medicinal plant from European Pharmacopea) were obtained by the proposed method and the content of verbascoside was determined at 40%. Each sample (1 ml) was placed in a 1 cm quartz cell. The absorbance was measured between 250-600 nm at an interval of every 1 nm using a Varian UV/Vis spectrophotometer (Cary 50 Scan). The following parameters were measured and calculated for each sample assuming 2 different application to the skin—2 mg/cm² and 4 mg/cm²: the SPF values, the Critical wavelength, and the UVA/UVB ratio.

Determination of SPF Values of the Aqueous Solutions of Lippia citriodora

Water solutions of Lippia citriodora extract at concentrations 0.002 and 0.001% were prepared in duplicate and UV-visible absorption spectra of were recorded over the range of 250-600 nm (FIG. 1.). From these spectra, the transmission of the sunscreen (T) was found from monochromatic absorbance values at wavelength λ: T=10^{-A(λ)}.

The SPF values of aqueous solutions of Lippia citriodora were calculated using Eq 1 for the lotions with 1.0%, 5.0%, 10.0%, 15.0% and 30.0% of Lippia citriodora extract applied at 2 mg/cm² (standard application) and 4 mg/cm².

TABLE 6
SPF values, critical wavelength (λ_{C}) and UVA/UVB
ratio for aqueous solutions of Lippia citriodora
with high concentrations of verbascoside (VB, 40%)

| | Final | | | | | |
|---|---|---|---|---|---|---|
| | concentration of | | | | | |
| | Lippia citriodora | | SPF values | | Critical | |
| | extract and | | 2 | 4 | wavelength | |
| No. | (verbascoside) | | mg/cm² | mg/cm² | (λ_{C}) | UVA/UVB |
| 5 | 1.0% | (0.4%) | 1.5 | 3.1 | 353 nm | 0.49 |
| 6 | 5.0% | (2.0%) | 4.8 | 29 | 353 nm | 0.49 |
| 7 | 10% | (4.0%) | 6.9 | 55 | 353 nm | 0.49 |
| 8 | 15.0% | (6.0%) | 26.0 | 124 | 353 nm | 0.49 |
| 9 | 30.0% | (12.0%) | 58.0 | 183 | 353 nm | 0.49 |

Critical Wavelength (λ_{C}) and the UVA/UVB ratios were also calculated from the absorption spectra and were collected in Table 6. According to Boots the Chemist Ltd, a low UVA protection corresponds to a UVA/UVB ratio <0.2 (0 UVA Star Rating), a moderate protection is at a ratio between 0.21 and 0.40 (1 UVA Star Rating), a good protection is at a ratio between 0.41 and 0.6 (2 UVA Star Rating), a superior protection is at a ratio between 0.61 and 0.8 (3 UVA Star Rating) and a maximum protection is at a ratio >0.8 (4 UVA Star Rating). According to these International indications, aqueous solutions of Lippia citriodora with high concentrations of verbascoside can be attributed to the group of Good protection (2 UVA Star Rating).

2. Photo-Stability of Aqueous Solutions of Lippia citriobara Extract Exposed to Solar Imitating UV Irradiation for 2-40 Minutes

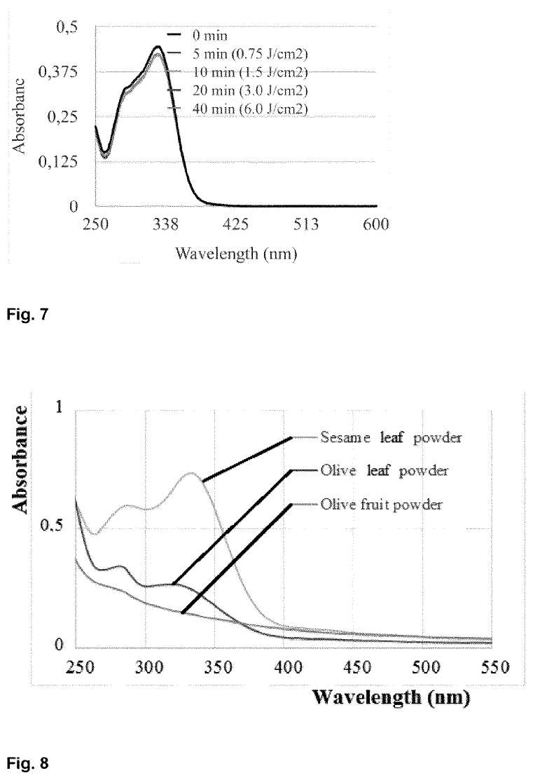

In these experiments, UV-visible absorption spectra of 0.002% aqueous solution of Lippia citriodora were recorded over the range of 250-450 nm without or after 2 to 40 min UV irradiation (FIG. 7). The total dose was 4.8 (UVB 1.6+UVA 3.2) J/cm². It was found that solutions are not destroyed by (UVA+UVB) light. UV irradiation did not affect SPF values of the solutions amid the absorption spectra even after 40 min of irradiation exhibit no changes (FIG. 7). It was found that Lippia citriodora extract was resistant to (UVA+UVB) light. Specifically, only very small influence of UV irradiation on absorption spectra of 0.002% water solutions of Lippia citriodora extract was found after 10 min of irradiation. However, the following irradiation (20 and 40 min) did not affect the absorption spectra (FIG. 7).

D. In Vitro Spectrophotometric Analysis of Sun Protection Factor (SPF, UVB), UVA Protection (UVA/UVB And Critical Wavelength), and Photo-Stability of Extracts of Sesamum indicum Leaves, Olea europea Leaves and Fruits

Extracts of leaves and fruit mash remained after olive oil extraction were prepared by different methods, where water or water: ethanol (1:1 V: V) or 96% ethanol were used. The extraction time was either shorter than the proposed (2 min) or the proposed (20-30 minutes) one. Samples of leaves and fruit mash (10 mg each) were diluted either by cold or hot (75° C.) water (5 ml) or by 5 ml of ethanol/water mixture or by 5 ml of pure ethanol (96%). The 0.2% suspensions were prepared in duplicates. Extraction procedure was carried out either manually for 2 min or by vortexing for 20-30 min. After extraction, solid unsolved matter was sedimented by centrifugation, supernatant collected, and the sediment was subjected to a secondary extraction in the same manner. Both supernatants obtained after the first and second extraction were pooled and used for the in vitro analyses. For spectrophotometry, water and ethanol suspensions were diluted by distilled water to concentration 0.02%, while water/ethanol extracts were diluted by distilled water to concentration 0.01%.

### 1. Determination of SPF Values of the Extracts

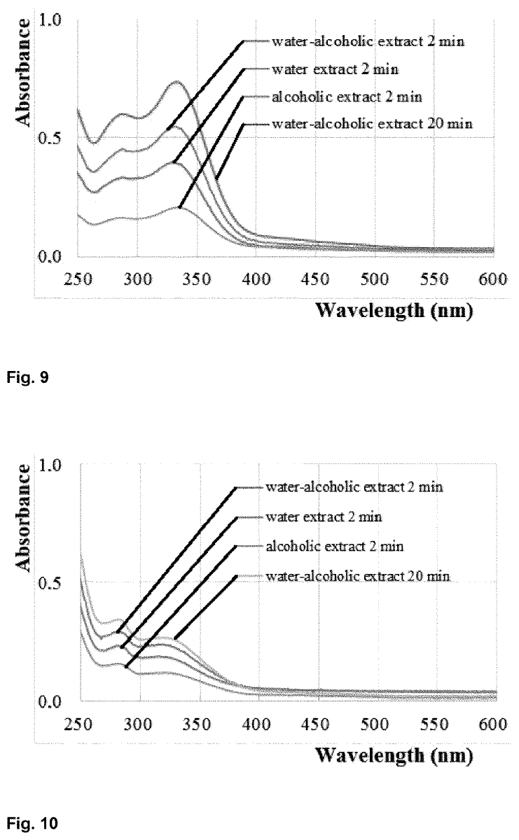

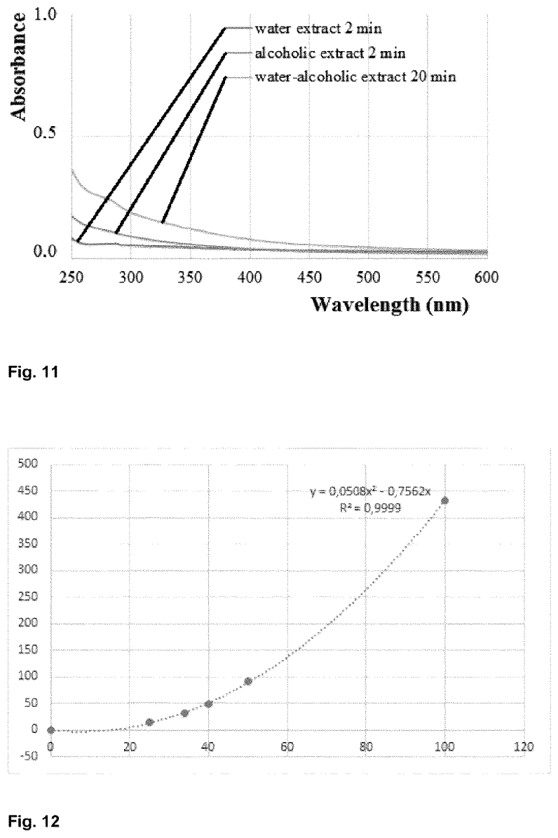

UV-visible absorption spectra of extracts prepared according to section “Sample preparation” were recorded over the range of 250-600 nm (examples are given in FIGS. 8-11). On the basis of these spectra, the transmission of the sunscreen (T) was found from monochromatic absorbance values at wavelength λ: T=10^{-A(λ)}.

SPF values were calculated using Eq. 1 for sun protective cosmetics applied at 2 mg/cm² (standard application).

TABLE 7
SPF values of sun protective lotions containing
extracts of Sesamum indicum leaves

| | Amount of ground sesame leaves used | | | |
|---|---|---|---|---|
| | for the extraction (g/100 ml solvent, %) | | | |
| Type of extraction | 10% | 25% | 50% | 100% |
| Water extraction by | 2.3 | 8 | 46 | 282 |
| hand shaking 2 min | | | | |
| Alcoholic extraction | 1.5 | 2.6 | 6.5 | 36 |
| by hand shaking 2 min | | | | |
| Water/alcoholic extraction | 3.2 | 16 | 117 | 600 |
| by hand shaking 2 min | | | | |
| Water/alcoholic extraction | 3.9 | 25 | 195 | 1040 |
| with vigorous shaking 20 min | | | | |

TABLE 8
SPF values for sun protective lotions containing
extracts of Olea europea leaves

| | Amount of ground olive leaves used | | | |
|---|---|---|---|---|
| | for the extraction (g/100 g solvent, %) | | | |
| Type of extraction | 10% | 25% | 50% | 100% |
| Water extraction by | 1.5 | 2.7 | 7.3 | 44 |
| hand shaking 2 min | | | | |
| Alcoholic extraction | 1.3 | 1.9 | 3.5 | 12 |
| by hand shaking 2 min | | | | |
| Water/alcoholic extraction | 1.7 | 3.6 | 12 | 91 |
| by hand shaking 2 min | | | | |
| Water/alcoholic extraction | 1.8 | 4.1 | 15 | 112 |
| with vigorous shaking 20 min | | | | |

TABLE 9
SPF values of sun protective lotions containing
extracts of Olea europea fruit mash

| | Amount of final extracts of | | | |
|---|---|---|---|---|
| | olive fruit mash used for the | | | |
| | extraction (g/100 g solvent, %) | | | |
| Type of extraction | 10% | 25% | 50% | 100% |
| Water extraction by | 1.1 | 1.3 | 1.8 | 3.2 |
| hand shaking 2 min | | | | |
| Alcoholic extraction | 1.2 | 1.6 | 2.5 | 6.0 |
| by hand shaking 2 min | | | | |
| Water/alcoholic extraction | 1.1 | 1.4 | 1.9 | 3.6 |
| by hand shaking 2 min | | | | |
| Water/alcoholic extraction | 1.5 | 2.6 | 6.6 | 41 |
| with vigorous shaking 20 min | | | | |

TABLE 10
Critical wavelength (λ_{C}) and UVA/UVB ratio for
sun protective products containing plant extracts

| | | Critical | |
|---|---|---|---|
| | | wavelength | |
| No. | Plant extracts | (λ_{C}) | UVA/UVB |
| 1 | Sesamum indicum leaf extract | 368 nm | 0.67 |
| 2 | Olea europea leaf extract | 373 nm | 0.56 |
| 3 | Olea europea fruit mash extract | 386 nm | 0.77 |

Critical Wavelength (λ_{C}) and UVA/UVB ratio of LOTIONs were also calculated on the base of absorption spectra and are given in tables 7 to 10. According to Boots the Chemist Ltd, a low UVA protection is obtained with a UVA/UVB ratio <0.2 (0 UVA Star Rating), a moderate protection with a ratio between 0.21 and 0.40 (1 UVA Star Rating), a good protection with a ratio between 0.41 and 0.6 (2 UVA Star Rating), a superior protection with a ratio between 0.61 and 0.8 (3 UVA Star Rating) and a maximum one with a ratio >0.8 (4 UVA Star Rating). Therefore, Olive leaf extract can be attributed to the group 2 UVA Star Rating while Olive fruit mash extract and Sesame leaf extract can be attributed to the group 3 UVA Star Rating.

Using spectra of different sesame leave extracts (FIG. 12), SPF values were calculated with the assumption of standard application of solutions containing sesame leave extracts to human skin as 2 mg/cm². The data are shown in Table 11.

TABLE 11
SPF values of the solutions containing
various extracts of sesame leave powder

| | Amount of sesame leaves (g) added | | | |
|---|---|---|---|---|
| | to 100 ml of a sun protective lotion | | | |
| Type of extraction | 10% | 25% | 50% | 100% |
| Water extraction by hand | 2.3 | 8 | 46 | 282 |
| shaking 2 min | | | | |
| Alcoholic extraction by hand | 1.5 | 2.6 | 6.5 | 36 |
| shaking 2 min | | | | |
| Water/alcoholic extraction by | 3.2 | 16 | 117 | 600 |
| hand shaking 2 min | | | | |
| Water/alcoholic extraction by | 3.9 | 25 | 195 | 1040 |
| vigorous shaking 20 min | | | | |
| Hot water extraction at 80-90° C. | 3 | 14 | 92 | 432 |
| and vigorous shaking 20 min | | | | |
| Hot water extraction at 80-90° C. | 3.1 | 15 | 94 | 363 |
| and vigorous shaking 30 min | | | | |

For the extraction by hot water with vigorous shaking for 20 min, which is the most promising type of extraction from the industrial point of view, the curve of SPF dependence on the initial amount of sesame leaves was produced (FIG. 12).
where X is amount of grounded sesame leaves and Y is the SPF value calculated by the Sayre equation.

From this exponential curve, it could be easily determined that:
100 g grounded sesame leaves added to 100 ml hot water will produce SPF 432
50 g grounded sesame leaves added to 100 ml hot water will produce SPF 92
40 g grounded sesame leaves added to 100 ml hot water will produce SPF 49
34 g grounded sesame leaves added to 100 ml hot water will produce SPF 31
25 g grounded sesame leaves added to 100 ml hot water will produce SPF 15

Sesamum indicum and Olea europea leaf extracts were extremely stable under exposure to solar light-imitating UVA+UVB irradiation for at least 40 min.

### E. In Vitro Determination of Sun Protective Properties of Chemical Sunscreens

### Chemical UV-Filters

1. Ethylhexyl methoxycinnamate, a liquid insoluble in water, (M.w. 290.4);
2. Benzotriazolyl dodecyl p-cresol, a liquid insoluble in water (M.w. 393.6);
3. Octocrylene, a liquid insoluble in water (M.w. 361.48);
4. Avobenzone (butylmethoxydibenzoylmethane), powder insoluble in water (M.w. 310.39);
5. Diethylhexyl butamido triazone, powder insoluble in water (M.w. 766.0;)
6. Ethylhexyl triazone, powder insoluble in water (M.w. 823.1);
7. Bis-ethylhexyloxyphenol methoxyphenyl triazine, powder insoluble in water (M.w. 627.8).

### Sample Preparation

### Liquid Water-Insoluble Sunscreens (Samples 1-3)

The 1st dilution: 50 mg of a liquid dissolved in 25 ml dimethyl sulfoxide (DMSO).
The 2nd dilution: 0.05 ml DMSO solution added to 10 ml water.
The 3d dilution: 0.05 ml DMSO/water solution added to 10 ml ethyl alcohol (96%).

### Powder Water-Insoluble Sunscreens (Samples 4-6)

The 1st dilution: 50 mg of powders were dissolved in 25 ml dimethyl sulfoxide (DMSO).
The 2nd dilution: 0.05 ml DMSO solution were added to 10 ml water.

### Powder Water-Insoluble and DMSO-Insoluble (Sample 7)

The 1st dilution: 50 mg of powder were dissolved in 25 ml heptane.
The 2nd dilution: 0.05 ml heptane solution were mixed with 5 ml heptane and incubated for 24 h

### For Determination of SPF, Critical Wavelength (λ_{C}) and UVA/UVB Ratio

The same methods and equations as in the case of plant extracts were applied.

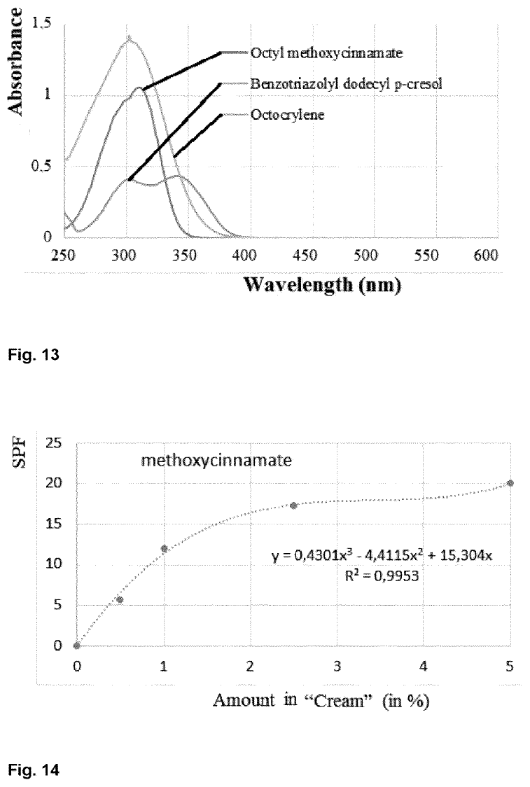

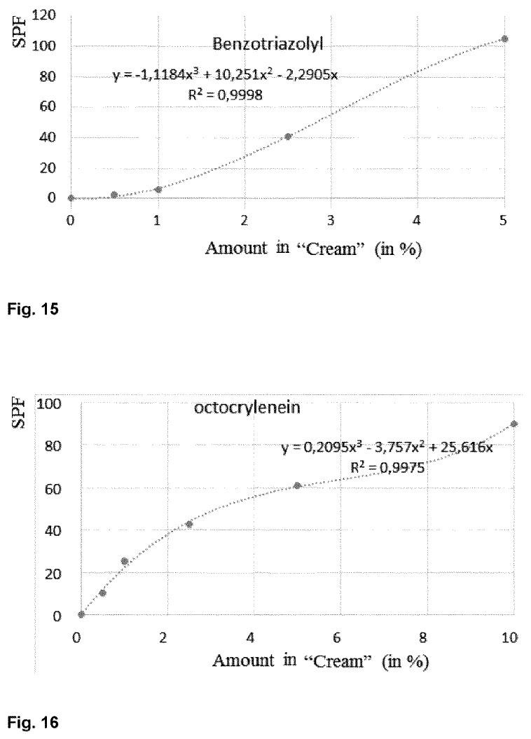

Liquid sunscreens. Since aqueous solutions of the samples were rather turbid and produce significant light scattering, DMSO/water/ethanol solutions were used for the calculation of SPF values (FIG. 13).

### 1. Determination of Sunscreen Capacity of Ethylhexyl Methoxycinnamate

Solutions of sunscreens at concentrations 0.001% were prepared in duplicate and UV-visible absorption spectra were recorded over the range of 250-600 nm (FIG. 1). On the basis of these spectra, the transmission of the sunscreen (T) was found from monochromatic absorbance values at wavelength λ: T=10^{-A(λ)}.

Table 12 shows dependence of SPF-B values on the content of octyl methoxycinnamate (%) in sun protective solutions and UV-A protection assessed by the ratio of UVA/UVB and critical wavelength. FIG. 2 shows a dose-dependent curve of SPF values from the chemical sunscreen concentration (%, octyl methoxycinnamate).

TABLE 12
SPF values, critical wavelength (λ_{C}) and
the UVA/UVB ratio for octyl methoxycinnamate.

| Amount of octyl | | Critical | |
|---|---|---|---|
| methoxycinnamate in sun | SPF | wavelength | |
| protective solution (%) | values | (λ_{C}) | UVA/UVB |
| 0.5% | 5.7 | 329 nm | 0.12 |
| 1% | 12 | 329 nm | 0.12 |
| 2.5% | 17 | 329 nm | 0.12 |
| 5% | 20 | 329 nm | 0.12 |
| 10% | 23 | 329 nm | 0.12 |
| 20% | 27 | 329 nm | 0.12 |

Conclusions: According to the in vitro spectrophotometry data obtained, the liquid chemical sunscreen octyl methoxycinnamate provides a dose-dependent SPF-B reaching SPF 20 at a concentration of 5%. A further increase to 10 and 20% led to a slight increase in the SPF values (23 and 27, respectively). The sunscreen does not possess good UV-A absorption in accordance with a low ratio of UVA/UVB equal to 0.12 and a critical wavelength of 329 nm

2. Determination of Sunscreen Capacity of Benzotriazolyl Dodecyl p-Cresol

Table 13 shows the dependence of SPF-B values on the content of benzotriazolyl dodecyl p-cresol (%) in sun protective solutions and UV-A protection assessed by the ratio of UVA/UVB and critical wavelength. FIG. 3 shows dose-dependent curve of SPF values.

TABLE 13
SPF values, critical wavelength (λ_{C}) and UVA/UVB
ratio for benzotriazolyl dodecyl p-cresol.

| Amount of benzotriazolyl | | Critical | |
|---|---|---|---|
| dodecyl p-cresol in sun | SPF | wavelength | |
| protective solution (%) | values | (λ_{C}) | UVA(UVB |
| 0.5% | 2.4 | 363 nm | 0.62 |
| 1% | 6 | 363 nm | 0.62 |
| 2.5% | 41 | 363 nm | 0.62 |
| 5% | 105 | 363 nm | 0.62 |
| 10% | 157 | 363 nm | 0.62 |

### Conclusion

The liquid sunscreen benzotriazolyl dodecyl p-cresol provides excellent dose-dependent UV-B protection that allows to reach an SPF-B 41 at a concentration of 2.5%. A further increase in its concentration up to 5 and 10% led to the SPF increase to 105 and 157, respectively. The sunscreen is also a very good protector from UV-A light having shown the ratio UVA/UVB equal to 0.62 and a critical wavelength at 363 nm

### 3. Determination of Sunscreen Efficiency of Octocrylene

Table 14 shows dependence of SPF-B values on the content of octocrylene (%) in sun protective solutions and UV-A protection assessed by the ratio of UVA/UVB and critical wave-length. FIG. 16 shows a dose-dependent curve of SPF values.

TABLE 14
SPF values, critical wavelength (λC)
and UVA/UVB ratio for octocrylene

| | | Critical | |
|---|---|---|---|
| Amount of octocrylene in | SPF | wavelength | |
| sun protective solution (%) | values | (λ_{C}) | UVA(UVB |
| 0.5% | 10 | 342 nm | 0.22 |
| 1% | 25 | 342 nm | 0.22 |
| 2.5% | 42 | 342 nm | 0.22 |
| 5% | 61 | 342 nm | 0.22 |
| 10% | 90 | 342 nm | 0.22 |

Conclusion: The liquid sunscreen octocrylene provides excellent dose-dependent UV-B protection that allows to reach a SPF-B 25 at the concentration 1.0%. A further increase in its concentration up to 2.5, 5.0 and 10.0% led to the SPF increase to 42, 61, and 90, respectively. However, the sunscreen does not protect from UV-A light having shown the ratio UVA/UVB equal to 0.22 and a critical wavelength at 342 nm

### Powder Sunscreens

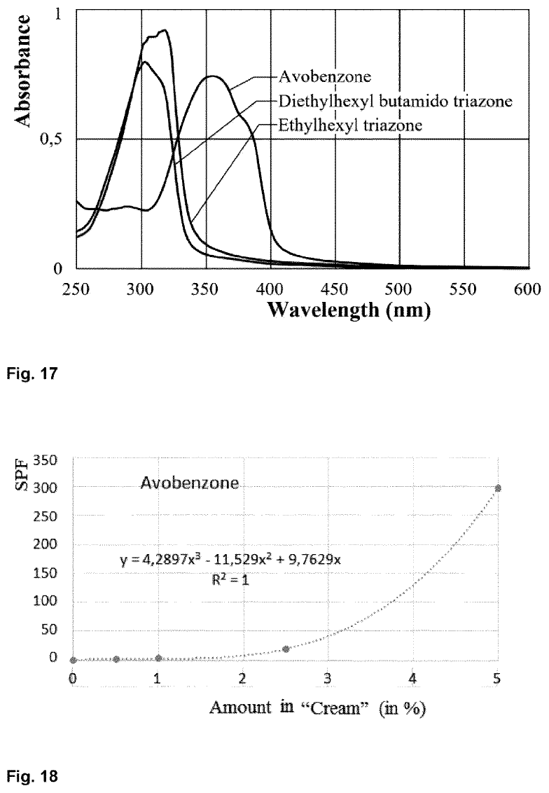

FIG. 17 shows UV-visible spectra of powder sunscreens (avobenzone, diethylhexyl butamido triazone, and ethyhexyl triazone).

### 4. Determination of Sunscreen Capacity of Avobenzone (Butylmethoxydibenzoyl-Methane)

Table 15 shows a dependence of SPF-B values on the content of avobenzone (%) in sun protective solutions and UV-A protection assessed by the ratio of UVA/UVB and critical wave-length, while FIG. 18 shows a dose-dependent curve of SPF values.

TABLE 16
SPF values, critical wavelength (λ_{C})
and UVA/UVB ratio for avobenzone

| | | | Critical | |
|---|---|---|---|---|
| | Amount of octocrylene in | SPF | wavelength | |
| No. | sun protective solution (%) | values | (λ_{C}) | UVA(UVB |
| 1 | 0.5% | 1.9 | 385 nm | 2.26 |
| 2 | 1% | 3.0 | 385 nm | 2.26 |
| 3 | 2.5% | 19 | 385 nm | 2.26 |
| 4 | 5% | 297 | 385 nm | 2.26 |

Conclusion: The powder sunscreen avobenzone provides moderate dose-dependent UV-B protection that allows to reach an SPF-B 19 at the concentration 2.5%. A further increase in its concentration up to 5.0% led to a sharp SPF increase to 297. However, the sunscreen is an excellent protector from UV-A light having shown the ratio UVA/UVB equal to 2.26 and a critical wavelength at 385 nm

### 5. Determination of Sunscreen Capacity of Diethylhexyl Butamido Triazone

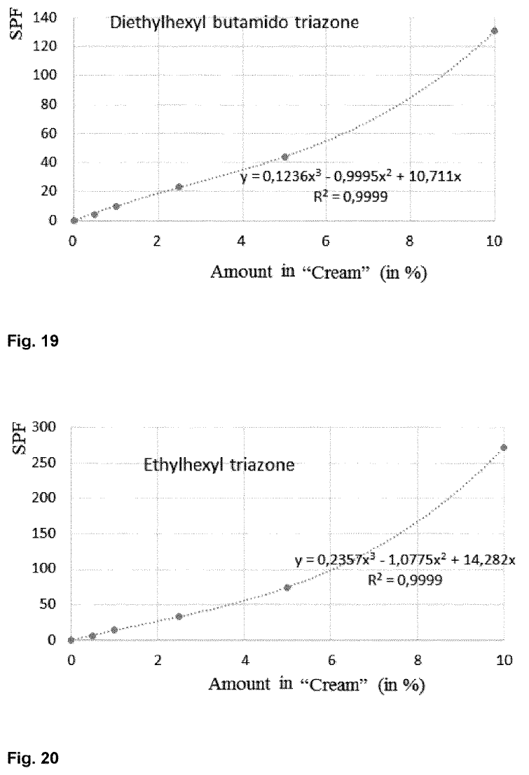

Table 17 shows a dependence of SPF-B values on the content of diethylhexyl butamido triazone (%) in sun protective solutions and UV-A protection assessed by the ratio of UVA/UVB and critical wavelength. FIG. 19 shows a dose-dependent curve of SPF values.

TABLE 17
SPF values, critical wavelength (λ_{C}) and the
UVA/UVB ratio for diethyl-hexyl butamido triazone

| Amount of diethylhexyl | | Critical | |
|---|---|---|---|
| butamido triazone in sun | SPF | wavelength | |
| protective solution (%) | values | (λ_{C}) | UVA(UVB |
| 0.5% | 4.1 | 337 nm | 0.14 |
| 1% | 10 | 337 nm | 0.14 |
| 2.5% | 23 | 337 nm | 0.14 |
| 5% | 44 | 337 nm | 0.14 |
| 10% | 131 | 337 nm | 0.14 |

Conclusion: The powder sunscreen ethylhexyl butamido triazone provides good dose-dependent UV-B protection that allows to reach an SPF-B of 23 at the concentration of 2.5%. A further increase in its concentration up to 5.0 and 10% led to the SPF increase to 44 and 137, respectively. The sunscreen practically does not protect from UV-A light having shown the ratio UVA/UVB equal to 0.14 and a critical wavelength at 337 nm

### 6. Determination of Sunscreen Capacity of Ethylhexyl Triazone

Table 18 shows dependence of SPF-B values on the content of ethylhexyl triazone (%) in sun protective solutions and UV-A protection assessed by the ratio of UVA/UVB and critical wave-length. FIG. 20 shows dose-dependent curve of SPF values.

TABLE 18
SPF values, critical wavelength (λ_{C})
and UVA/UVB ratio for ethylhexyl triazone

| | | Critical | |
|---|---|---|---|
| Amount of ethyl hexyl triazone | SPF | wavelength | |
| in sun protective solution (%) | values | (λ_{C}) | UVA(UVB |
| 0.5% | 5.2 | 341 nm | 0.21 |
| 1% | 14 | 341 nm | 0.21 |
| 2.5% | 33 | 341 nm | 0.21 |
| 5% | 74 | 341 nm | 0.21 |
| 10% | 271 | 341 nm | 0.21 |

Conclusion: The powder sunscreen ethyl hexyl triazone provides very good dose-dependent UV-B protection that allows to reach an SPF-B of 44 at the concentration of 2.5%. A further increase in its concentration up to 5.0 and 10% led to the SPF increase to 74 and 271, respectively. The sunscreen is a moderate protector from UV-A light having shown the ratio UVA/UVB equal to 0.21 and a critical wavelength at 341 nm

### 7. Determination of Sunscreen Capacity of Bis-Ethylhexyloxyphenol Methoxyphenyl Triazine

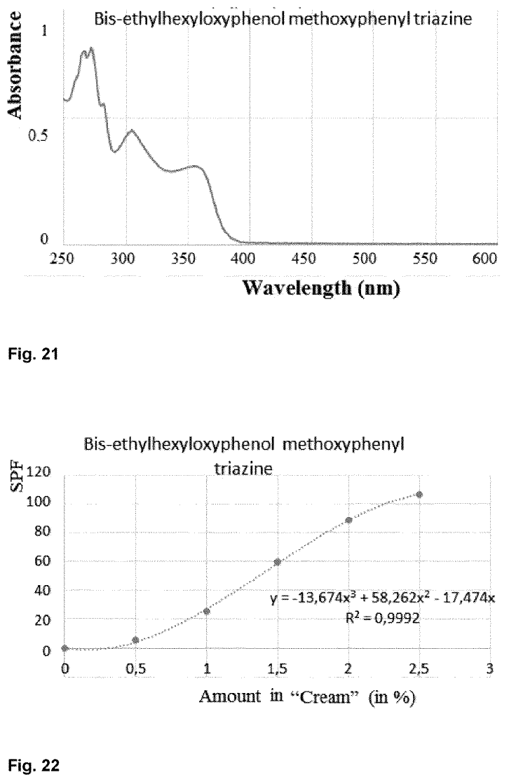

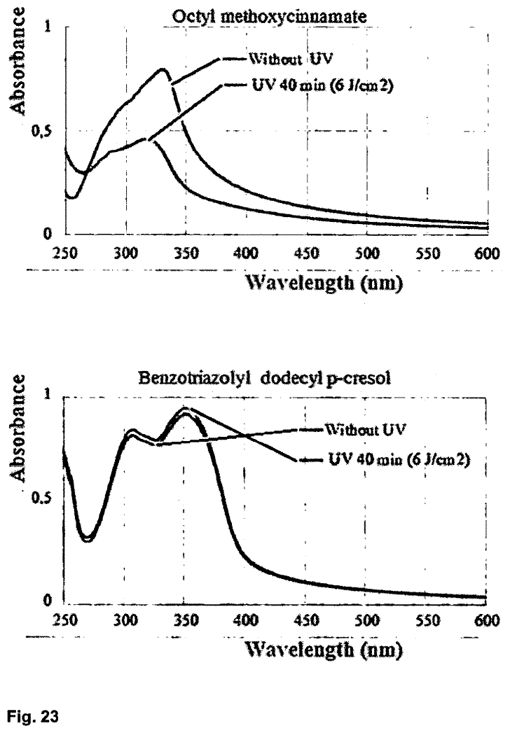

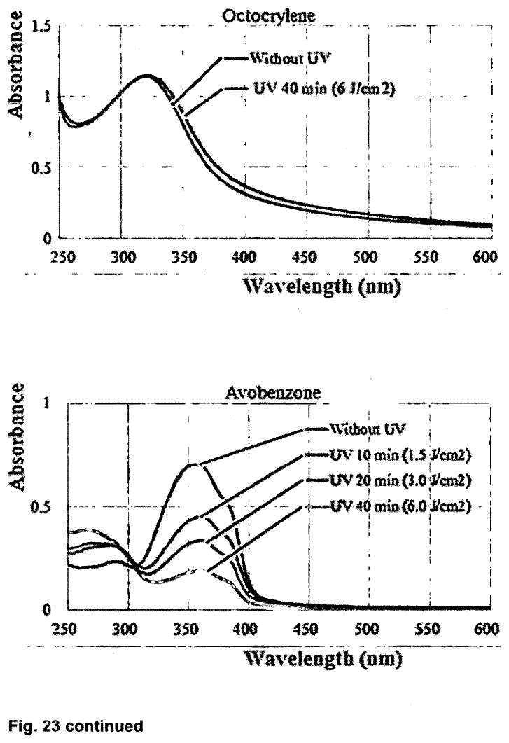

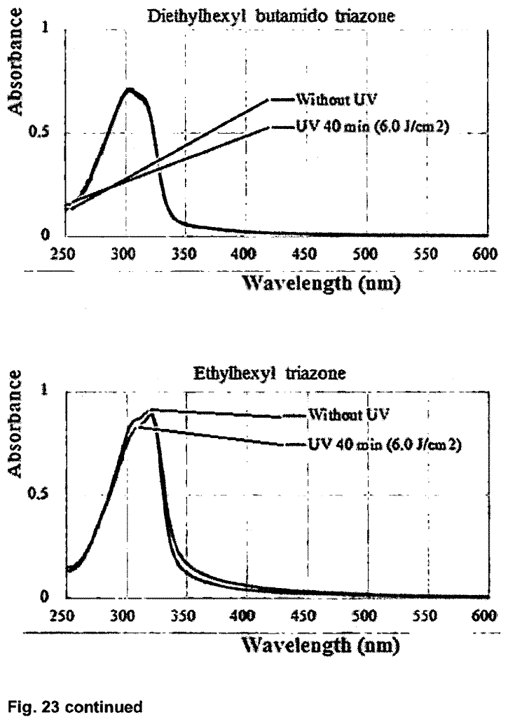

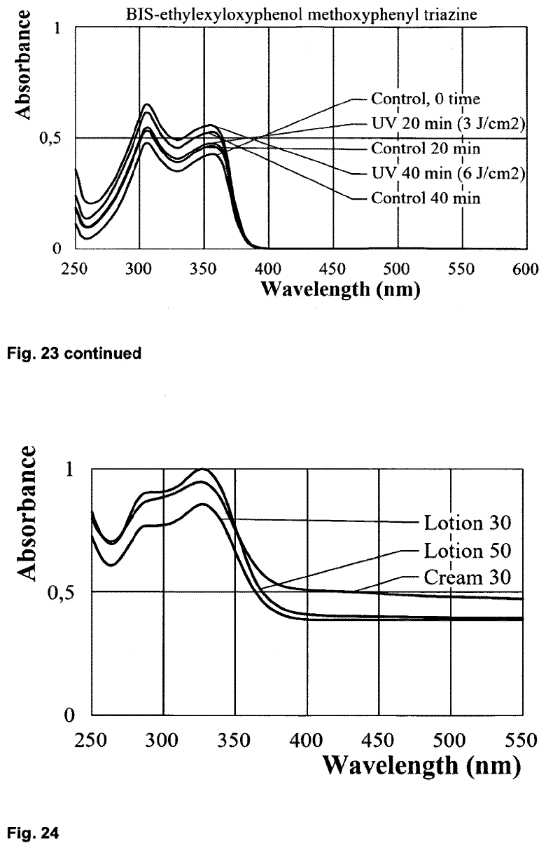

This substance was not dissolved in ethyl and isopropyl alcohols, dimethyl sulfoxide. It was possible to obtain a solution only in heptane, after several days of incubation in solvent. FIG. 21 shows the UV-visible spectrum of the substance dissolved in heptane.

Table 19 shows the dependence of SPF-B values on the content of bis-ethylhexyloxyphenol methoxyphenyl triazine (%) in sun protective solutions and UV-A protection assessed by the ratio of UVA/UVB and critical wavelength. FIG. 22 shows a dose-dependent curve of SPF values.

TABLE 19
SPF values, critical wavelength (λ_{C}) and UVA/UVB
ratio for bis-ethylhexyloxyphenol methoxyphenyl triazine

| Amount of bis- | | | |
|---|---|---|---|
| ethylhexyloxyphenol | | Critical | |
| methoxyphenyl triazine in | SPF | wavelength | |
| sun protective solution (%) | values | (λ_{C}) | UVA(UVB |
| 0.5% | 5.7 | 365 nm | 0.50 |
| 1% | 25 | 365 nm | 0.50 |
| 2.5% | 60 | 365 nm | 0.50 |
| 5% | 89 | 365 nm | 0.50 |
| 10% | 107 | 365 nm | 0.50 |

Conclusion: The powder sunscreen bis-ethylhexyloxyphenol methoxyphenyl triazine provides very good dose-dependent UV-B protection that allows to reach an SPF-B of 25 at the concentration of 1.0%. A further increase in its concentration up to 1.5, 2.0, and 2.5% led to the SPF increase to 60, 89, and 107, respectively. The sunscreen is a good protector from UV-A light having shown the ratio UVA/UVB equal to 0.50 and a critical wavelength of 365 nm.

### Determination of the Photo Resistance of Chemical and Physical Sunscreens to UVA+UVB Irradiation

Susceptibility of water solutions of chemical sunscreens to UV irradiation was followed spectrophotometrically. Samples (3 ml) of water solution with concentrations of 0.001% were exposed to 2 mW/cm² UV light (G6T5E UV-B lamp, emitted ultraviolet rays between 280 nm and 360 nm (at peak 306 nm) UVB 0.7 mW/cm²+UVA 1.3 mW/cm²) in 3.5-cm Petri dishes. The distance from the surface was 5.5 cm, the layer thickness was ˜ 2 mm. The lengths of exposure was up to 40 min. Irradiance was measured using UVX Digital Radiometer equipped with UVX-31 300 nm and UVX-36 365 nm sensors (Canadawide Scientific). Then, the absorbance was measured between 250-600 nm using a 1 cm quartz cell. The control samples were treated in the same manner except UV irradiation.

### Conclusion

Octyl methoxy cinnamate, avobenzone, and bis-ethylhexyloxyphenol methoxyphenyl triazine are HIGHLY unstable to solar-imitating UV irradiation. The other chemical sunscreens studied were as stable to strong UV irradiation (40 min; 6 J/cm²) as the sunscreen natural plant extracts.

General conclusions: Chemical s approved worldwide and given the exclusivity of being used as SPF-B and SPF-A in sun protective cosmetics possess several evident disadvantages as compared to plant-derived sunscreens based on verbascoside and its close analogues: (1) Being water-insoluble, they are soluble in the skin lipids, thus bearing risks of local (skin) and generalized (whole organism) toxicity including embryo-toxicity, teratogenesis, hormonal disruption, carcinogenesis, and allergenicity; (2) Some of them being susceptible to destruction by solar UV irradiation, should be frequently substituted by another application to the skin; (3) Metabolites of photo-destroyed chemical sunscreens are highly toxic and photo-toxic to human skin and organism in general, and to aqueous, marine and terrestrial living organisms, microorganisms, plants and animals alike, such as weeds, algae, plankton, fish, clams, crabs, turtles, to name a few. Therefore, their negative impact to environment is immense; (4) Great majority of chemical sunscreens selectively protect either from UVA or from UVB light and only few of them belong to a broadband UV screens. Therefore, usually cosmetic sun protective compositions should contain a combination of several different chemical sunscreens to achieve full UVA+UVB protection prescribed by dermatologists/cosmetologists to prevent premature skin ageing and skin cancers.

### Comparative Data on SPF-B of Synthetic Chemical Sunscreens and Plant Derived Verbascoside- or ITA Analogues-Containing Sunscreens

In the following Table 20, there are SPF values obtained in vitro spectrophotometrically, which have been calculated by a classical Sayre method (Sayre, R M; Agin, P P; LeVee, G J; Marlowe, E. A comparison of in vivo and in vitro testing of sunscreening formulas, Photochem. Photobiol. 1979, 29, 559-566) and modified Sayre method, which gives results similar to those obtained in the in vivo tests recommended by COLIPA.

| | | Amount of UV filters in sun protective | | | | | |
|---|---|---|---|---|---|---|---|
| | | cosmetic (%) | | | | | |
| | Calculated by the | 0.5 | 1.0 | 2.5 | 5 | 10 | 20 |
| n/n | Equations | SPF values | | | | | |
| 1 | Oxyl methoxycinnamate | | | | | | |
| | Sayre | 4.3 | 10 | 15.5 | 17 | 19 | 20 |
| | Sunscreen simulator | 2 | 2.8 | 4.9 | 7.9 | 12.4 | 18.8 |
| | Modified Sayre | 4.1 | 6.3 | 11 | 17 | 26 | 40 |
| 2 | Benzotriazolyl dodecyl p-cresol | | | | | | |
| | Sayre | 2.1 | 5 | 29 | 91 | 132 | |
| | Modified Sayre | 2.0 | 3.1 | 5.4 | 8.3 | 12.7 | 19 |
| 3 | Ocrylene | | | | | | |
| | Sayre | 1.9 | 4 | 13 | 26.5 | 36.8 | |
| | Sunscreen simulator | 1.6 | 2.1 | 3.5 | 6 | 11 | |
| | Modified Sayre | 1.6 | 2.6 | 4.7 | 7.4 | 11.7 | |
| 4 | Avobenzone (butylmethoxydibenzoylmethane) | | | | | | |
| | Sayre | 1.5 | 2.0 | 6.1 | 32 | | |
| | Sunscreen simulator | 1.5 | 1.9 | 3.0 | 4.5 | | |
| | Modified Sayre | 1.5 | 2.1 | 3.2 | 4.4 | | |
| 5 | Diethylhexyl butamido triazone | | | | | | |
| | Sayre | 3.2 | 9.0 | 39 | 98 | 394 | |
| | Sunscreen simulator | 2.4 | 3.3 | 5.7 | 8.3 | 11.1 | |
| | Modified Sayre | 3.2 | 4.6 | 7.4 | 10.6 | 15.3 | |
| 6 | Ethylhexyl triazone | | | | | | |
| | Sayre | 2.8 | 8 | 69 | 389 | | |
| | Sunscreen simulator | 2.4 | 3.4 | 5.9 | 8.7 | N/D | |
| | Modified Sayre | 2.8 | 4.1 | 6.8 | 10.1 | | |
| 7 | Bis-ethylhexyloxyphenol methoxyphenyl triazine (hexane) | | | | | | |
| | Sayre | 4.8 | 19 | 93 | | | |
| | Sunscreen simulator | 2.1 | 3.1 | 6.1 | 12.0 | 25.1 | |
| | Modified Sayre | 4.2 | 7.5 | 16.1 | 29 | 50 | |

| | | Amount of verbascoside or its analogues | | | | | | | |
|---|---|---|---|---|---|---|---|---|---|
| | | derived from plant/plant cell culture | | | | | | | |
| | | extracts in sun protective cosmetic (%) | | | | | | | |
| | Calculated by the | 0.5 | 1.0 | 2.5 | 5 | 8 | 10 | 12 | 20 |
| n/n | Equations | SPF values | | | | | | | |
| 1 | Cistanche tubulosa | | | | | | | | |
| | Sayre | 1.6 | 2.6 | 9.0 | 33 | — | 64 | — | 97 |
| | Modified Sayre | 1.5 | 2.6 | 5.6 | 10 | — | 18 | — | 31 |
| | SPF = 2.63* | | | | | | | | |
| | exponentiation | | | | | | | | |
| | (X; 0.827) | | | | | | | | |
| 2 | Lippia citriodora | | | | | | | | |
| | Sayre | 1.5 | 2.4 | 8.0 | 31 | — | 59 | — | 83 |
| | Modified Sayre | 1.4 | 2.5 | 5.3 | 9.4 | — | 17 | — | 30 |
| | SPF = 2.486* | | | | | | | | |
| | exponentiation | | | | | | | | |
| | (X; 0.827) | | | | | | | | |
| 3 | Verbascoside | | | | | | | | |
| | Sayre | 2.0 | 3.9 | 23 | 110 | | | | |
| | Modified Sayre | 2.2 | 3.9 | 8.3 | 14.8 | 22 | 26 | 31 | 52 |
| | SPF = 3.9* | | | | | | | | |
| | exponentiation | | | | | | | | |
| | (X; 0.827) | | | | | | | | |
| 4 | Verbascoside/Myconoside | | | | | | | | |
| | Sayre | 2 | 4 | 23 | 137 | | | | |
| | Modified Sayre | 2.3 | 4.1 | 8.7 | 15.5 | — | 28 | — | 53 |
| | SPF = 4.1* | | | | | | | | |
| | exponentiation | | | | | | | | |
| | (X; 0.827) | | | | | | | | |
| 5 | Extracts from Haberlea rhodopensis | | | | | | | | |
| | Sayre | 1.25 | 1.6 | 3.0 | 11.0 | — | 60 | — | — |
| | Modified Sayre | 0.9 | 1.7 | 3.5 | 6.3 | — | 11 | 27 | 49 |
| | SPF = 1.66* | | | | | | | | |
| | exponentiation | | | | | | | | |
| | (X; 0.827) | | | | | | | | |

Extracts from Sesamum indicum leaves
Water/alcoholic extraction with vigorous shaking 20 min

| Verbascoside content | 1.0% | 2.5% | 5.0% | 10% | 15% | 20% |
|---|---|---|---|---|---|---|
| Sayre | 3.9 | 25 | 195 | — | — | — |
| Modified Sayre | 4.1 | 8.7 | 15.5 | 28 | 39 | 58 |
| SPF = 4.1* | | | | | | |
| exponentiation | | | | | | |
| (X; 0.827) | | | | | | |

### X—Content of Sun Protective Substance in the Product

### X—Content of Sun Protective Substance in the Product

### Conclusions

(1) For water insoluble chemical filters, the modified Sayre method gives results close to those calculated by Simulator-Calculator (BASF) and the in vivo tests. The Simulator-Calculator uses the modified Sayre method assuming an exponentiation equal to 0.827 (X^{0.827}).
(2) For water soluble natural filters, the same modified Sayre method would give results close to those obtained by the in vivo tests (predicted by Simulator-Calculator).
   Examples of Cosmetic Compositions with Fully Natural Sun Protective Factors
1. Sun protective lotion SPF 50+, high UV-A protection
2. Sun protective cream SPF 50+, high UV-A protection
3. Sun protective serum for pregnant and milking women SPF 50+, high UV-A protection
4. Sun protective milk for babies SPF 50+, high UV-A protection
5. Sun protective lotion SPF 20-30, high UV-A protection for people with highly sensitive skin
   E. Analysis of Sun Protective Parameters of Two Lotions and Cream Containing Lippia citriodora Extract with 40% of Verbascoside.

### Sample Preparation

Samples were dissolved in water-ethanol mixture by the following procedure:
samples (approximately 200-300 mg) were dissolved in distilled water to 10 mg/ml, and then 0.5 ml of this mixture was diluted by 9.5 ml of ethanol (96%), to 0.5 mg/ml solutions in duplicate.

### Registration of Absorption Spectra

UV-visible absorption spectra of water/ethanol solutions were recorded over the range of 250-600 nm (FIG. 24).

These spectra were used for the calculation of the sunscreen transmission (T), which was found from monochromatic absorbance values at wavelength λ:

$$T = {10}^{- A ( λ )}$$

The SPF in vitro was calculated according to the method described by Sayre et al., (Sayre, R M; Agin, P P; LeVee, G J; Marlowe, E. A comparison of in vivo and in vitro testing of sunscreening formulas, Photochem. Photobiol. 1979, 29, 559-566) following equation 1:

$$SPF = {\sum }_{290}^{400} Ser ( λ ) \times Ss ( λ ) / {\sum }_{290}^{400} Ser ( λ ) \times Ss ( λ ) \times T ( λ ) , \qquad ( 1 )$$

where Ss(λ) is the spectral irradiance, Ser(λ) the CIE erythema action spectrum and T(λ) the trans-mission of the sunscreen. Data for Ss(λ) and Ser(λ) are available in the literature (B. L. Diffey, J. Robson. A new substrate to measure sunscreen protection factors throughout the ultraviolet spectrum, J. Soc. Cosmet. Chem. 1989, 40, 127-133).

The Critical Wavelength λ_{C} value for the test product was defined as that wavelength where the area under the absorbance spectrum of the sample from 290 nm to λ_{C} is 90% of the integral of the absorbance spectrum from 290 to 400 nm and is calculated using Eq. 2:

$${\int }_{290}^{λ c} A ( λ ) d λ = 0.9 {\int }_{290}^{400} A ( λ ) d λ \qquad ( 2 )$$

A(λ)=monochromatic absorbance values at wavelength λ

In order to characterize the ability of creams to protect skin against UVA the UVA/UVB ratio was calculated using Eq. 3:

$$UVA / UVB = {\int }_{320}^{400} A ( λ ) d λ / {\int }_{290}^{320} A ( λ ) d λ \qquad ( 3 )$$

The SPF values were calculated using Eq 1 with the application conditions 2 mg/cm² (Standard application recommended by the European Agency).

TABLE 21
SPF values, critical wavelength (λ_{C}) and UVA/UVB ratio

| | | SPF values | Critical | |
|---|---|---|---|---|
| | Sun protective | calculated | wavelength | |
| No. | products | from spectra | (λ_{C}) | UVA/UVB |
| 1 | Lotion SPF 30 | 29.7 | 361 nm | 0.68 |
| 2 | Lotion SPF 50 | 52 | 361 nm | 0.72 |
| 3 | Cream SPF 50 | 49.8 | 357 nm | 0.67 |

Critical Wavelength (λ_{C}) and UVA/UVB ratios were also calculated using Equation 2 and 3 (Table 21). According to Boots the Chemist Ltd, a low UVA protection is obtained with a UVA/UVB ratio <0.2 (0 UVA Star Rating), a moderate protection with a ratio between 0.21 and 0.40 (1 UVA Star Rating), a good protection with a ratio between 0.41 and 0.6 (2 UVA Star Rating), a superior protection with a ratio between 0.61 and 0.8 (3 UVA Star Rating) and a maximum one with a ratio >0.8 (4 UVA Star Rating). Therefore, Lotions and cream can be attributed to the group 3 UVA Star Rating.

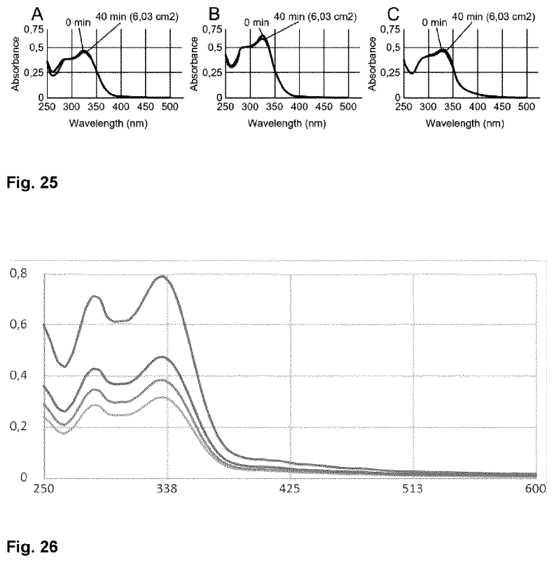

Susceptibility of water/alcohol solutions of the 2 lotions and the cream to UV irradiation was determined spectrophotometrically. Samples (3 ml) of water/alcohol solution with concentrations 0.5 mg/ml were exposed to 2 mW/cm² UV light imitating sun light (G6T5E UV-B lamp, emitted ultraviolet rays between 280 nm and 360 nm (at peak 306 nm) UVB 0.7 mW/cm²+UVA 1.3 mW/cm²) in 3.5-cm Petri dishes. The distance from the surface was 5.5 cm, the layer thickness was =2 mm. The lengths of exposure was 5-40 min. Irradiance was measured using UVX Digital Radiometer equipped with UVX-31 300 nm and UVX-36 365 nm sensors (Canadawide Scientific). Then, the absorbance was measured between 250-600 nm using a 1 cm quartz cell. The control non-irradiated samples were treated in the same manner. FIG. 25 shows negligible changes in the spectra after 40 min of irradiation (FIG. 25).

### Conclusions

1. Cosmetics (lotions and light cream) could reach SPF values as high as 30 and 50 due to the presence of natural SPF bearing substances, in this case, verbascoside from Lippia citriodora aqueous extract
2. These cosmetics with natural SPF strongly protect from UVA solar irradiation as was assessed by the critical wavelength and by the ratio UVA/UVB. In accord with the International Standards, these cosmetics could be considered as a superior protection with the UVA/UVB ratio between 0.61 and 0.8 (3 UVA Star Rating)
3. These cosmetics possess extremely high photo-stability as they are not destroyed by solar light imitating and long lasting (at least 40 min) exposure to UVA+UVB irradiation. Therefore they should not be frequently re-applied due to inactivation by solar UV irradiation
   F. Analysis of Sun Protective Parameters of Lotions Containing a Mixture of Syringa vulgaris and Haberlea rhodopensis Cultured Cell Extracts Containing Verbascoside (10%) and Myconoside (36%), Respectively.

Goal of the study was to evaluate in vitro UV protective properties of anti-solar lotions consisting of basic excipient-containing lotion (MEDENA AG is an owner of the formulation) and active ingredients coming from extracts of cultured meristem cells of Syringa vulgaris and Haberlea rhodopensis. The extract of Syringa vulgaris cultured cells was enriched with glycosylated phenyl propanoid verbascoside (10%) with well-known UVA+UVB photo-protective properties. The extract of cultured Haberlea rhodopensis cells used in the lotions contained 36% of phenyl propanoid myconoside. For the in vitro studies, spectrophotometric methods to predict results of future human studies corresponding the requirements of EU Commission for sun protective cosmetics (COLIPA) were applied (B. L. Diffey, J. Robson. A new substrate to measure sunscreen protection factors throughout the ultraviolet spectrum, J. Soc. Cosmet. Chem. 1989, 40, 127-133).

### 1. Determination of SPF Values of the Lotions

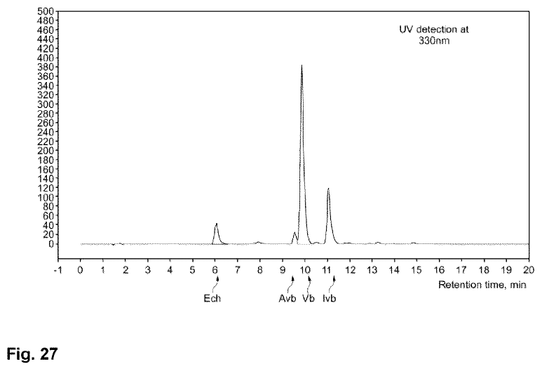

A series of verbascoside+myconoside-containing lotions containing the lotion excipients and a mixture of plant meristem cell extracts (of 5%/5%, 4%/4%, 3.5%/3.5% and 2%/2%, respectively) were prepared in duplicate using extracts from Haberlea rhodopensis (360 mg myconoside/g) and Syringa vulgaris (10% verbascoside). The UV-visible absorption spectra were recorded over the range of 250-600 nm (FIG. 26). From these spectra, the transmission of the sunscreen (T) was found from monochromatic absorbance values at wavelength λ: T=10^{-A(λ)}.

The SPF value for lotions were calculated using Eq. 1 for three application conditions at 2 mg/cm² (standard application), 2.5 mg/cm² and 3 mg/cm². In the same Table 22, critical wavelength and the UVA/UVB ratios are shown.

TABLE 22

| | Concentra- | | | | | |
|---|---|---|---|---|---|---|
| | tion of | | | | | |
| | verbascoside + | | | | Critical | |
| | myconoside | SPF values at different | | | wave- | |
| | mixture (%) | applications to the skin | | | length | UVA/ |
| No. | in lotion | 2 mg/cm² | 2.5 mg/cm² | 3 mg/cm² | (λ_{C}) | UVB |
| 1 | 2% | 15 | 33 | 42 | 363 nm | 0.65 |
| | verbascoside + | | | | | |
| | 2% | | | | | |
| | myconoside | | | | | |
| 2 | 3.5% | 23 | 42 | 66 | 363 nm | 0.65 |
| | verbascoside + | | | | | |
| | 3.5% | | | | | |
| | myconoside | | | | | |
| 3 | 4.0% | 42 | 74 | 112 | 363 nm | 0.65 |
| | verbascoside + | | | | | |
| | 4.0% | | | | | |
| | myconoside | | | | | |
| 4 | 5% | 137 | — | — | 363 nm | 0.64 |
| | verbascoside + | | | | | |
| | 5% | | | | | |
| | myconoside | | | | | |

SPF values, critical wavelength (λ_{C}) and UVA/UVB ratio for the “lotions” on the base of extracts from Haberlea rhodopensis (360 mg myconoside/g) and verbascoside powder (10% verbascoside) with different concentrations of verbascoside/myconoside

The critical Wavelength (λ_{C}) and the UVA/UVB ratios were also calculated from the absorption spectra and were collected in Table 22. According to Boots the Chemist Ltd, a low UVA protection corresponds to a UVA/UVB ratio <0.2 (0 UVA Star Rating), a moderate protection is at a ratio between 0.21 and 0.40 (1 UVA Star Rating), a good protection is at a ratio between 0.41 and 0.6 (2 UVA Star Rating), a superior protection is at a ratio between 0.61 and 0.8 (3 UVA Star Rating) and a maximum protection is at a ratio >0.8 (4 UVA Star Rating). According to these International indications, lotions containing a mixture of verbascoside and myconoside possess a Superior Protection (3 UVA Star Rating).

### Conclusions

(1) Protection from UVB irradiation reached SPF 23 and SPF 42 at standard 2.0 mg/cm² application mode when lotions contained 3.5/3.5% and 4.0/4.0% of phenyl propanoid-containing mixtures.
(2) Protection from UVA irradiation assessed by the UVA/UVB ratio corresponds to Superior Protection UVA 3 Star Rating (in accordance with the international standards) for the whole range of phenyl propanoids studied.
(3) Photo-stability of lotions is very high and equal to photo-stability of each of the two phenyl propanoids studied. These unique natural photo-protectors were not destroyed by intensive solar-imitating UVB+UVA irradiation for at least 40 minutes. So the application of verbascoside+myconoside-containing sun protective products should not be repeated very often.

1. A composition comprising an extract containing verbascoside and/or derivatives thereof and/or structural analogs thereof, wherein the extract is prepared from aerial parts of plants selected from the group consisting of sesame plant (Sesamum indicum), Lippia citriodora, Haeberlea rhodopensis, Cistanche tubulosa, Syringa vulgaris, Aiuga reptens, Buddleija davidii, Verbena officinalis, and Olea europea or wherein the extract is prepared from cell callus cultures of said parts of plants.

2. The composition according to claim 1, comprising 6.6 to 25 wt-% verbascoside and/or derivatives thereof and/or structural analogs thereof.

3. The composition according to claim 1, comprising at least one compound selected from the group consisting of amino acids, fatty acids, polysaccharides, sterols, vitamins, minerals, and phytochemical compounds.

4. The composition according to claim 1 in the form of a cream, a milky emulsion or a transparent lotion or a serum or a spray.

5. The composition according to claim 1, wherein the composition has an in-vitro SPF equal to or higher than 20.

6. The composition according to claim 1, wherein the verbascoside is embedded by liposomes, lipogels, hydrogels, nanoparticles or any other intracutaneous carrier.

7. The composition according to claim 1, wherein the composition has water resistance and adhesion to the skin surface.

8. A topic sunscreen against UV-A and UV-B radiation comprising a composition according to claim 1.

9. A method for obtaining an extract comprising verbascoside, derivatives thereof and/or structural analogs thereof, the method comprising the steps of:
a) selecting at least one aerial part or a callus cell culture of a plant selected from the group consisting of sesame plant (Sesamum indicum), Lippia citriodora, Haeberlea rhodopensis, Cistanche tubulosa, Syringa vulgaris, Aiuga reptens, Buddleija davidii, Verbena officinalis, and Olea europea;
b) extracting verbascoside, derivatives thereof and/or structural analogs thereof present in the plant part via a technique selected from the group consisting of washing, decoction, maceration, homogenization, percolation, and any combination thereof;
c) separating the main liquid phase which comprises the extracted compounds from the solids greater than approximately 2 mm in size by natural sedimentation, filtration, centrifugation or a combination thereof;
d) clarifying the liquid phase obtained in step c); and
e) concentrating the clarified liquid phase.

10. The method according to claim 9, comprising an additional step of pretreating the aerial plant part before the extraction step b), wherein the pretreatment comprises drying the aerial plant part to a moisture content of less than 80% to 60% and/or chopping the plant waste part to a size less than 5 cm.

11. The method according to claim 9, wherein the extraction step is carried out in water or a solvent/water mixture, wherein the solvent is chosen from the group consisting of methanol, ethanol, acetone, and ethyl acetate.

12. The method according to claim 9, wherein the extraction step is carried out at a temperature between 25° C. and 90° C.

13. The method according to claim 9, wherein the solids separated out in step c) are subjected to a second extraction under the same conditions as in the extraction performed in step b), wherein the secondary liquid phase is separated from the solids greater than 2 mm in size, and wherein the secondary liquid phase is mixed with the main liquid phase previously obtained.

14. The method according to claim 9, comprising an additional step f), in which the concentrated liquid from step e) is transformed into powder via a spray-drying technique.

15. The method according to claim 9, wherein the aerial plant parts are plant waste derived from the industrial manufacturing of plant-based food products, such as sesame oil, olive oil or sesame bran.

16. The composition according to claim 1, wherein the composition is a cosmetic formulation.

17. The method of claim 9, wherein the extract is selected from the group consisting of teupolioside, isoverbascoside, 2′-acyl-verbascoside, echinacoside, myconoside, tubuloside C, cistanoside A, and cistanoside C.

18. The method claim 12, wherein the extraction step is carried out for 20 to 60 minutes.

19. The method of claim 12, wherein the extraction step is carried out under continuous magnetic stirring or vortexing.
